SEQUENCE LISTING (20 sequences)

SEQ ID NO: 1 - DNA, 24 bp, artificial sequence
  synthetic construct
ggtcccacagacttaccaaactca

SEQ ID NO: 2 - DNA, 24 bp, artificial sequence
  synthetic construct
tcgactatggaccttagcacccat

SEQ ID NO: 3 - DNA, 30 bp, artificial sequence
  synthetic construct
ccgaatgtcgatgagtacagcatagcagac

SEQ ID NO: 4 - DNA, 24 bp, artificial sequence
  synthetic construct
ggatatagctggttctccgcgaaa

SEQ ID NO: 5 - DNA, 24 bp, artificial sequence
  synthetic construct
tcgaaggtagagcactgaatgagc

SEQ ID NO: 6 - DNA, 28 bp, artificial sequence
  synthetic construct
accatcgaaggtagagcactgaatgagc

SEQ ID NO: 7 - DNA, 28 bp, artificial sequence
  synthetic construct
gggtcccacagacttaccaaactcaatc

SEQ ID NO: 8 - DNA, 23 bp, artificial sequence
  synthetic construct
caaactccgaatgtcaataaagt

SEQ ID NO: 9 - DNA, 20 bp, artificial sequence
  synthetic construct
atgacgatctgggctgtttc

SEQ ID NO: 10 - DNA, 20 bp, artificial sequence
  synthetic construct
tgtgggcgctaaggttcata

SEQ ID NO: 11 - DNA, 19 bp, artificial sequence
  synthetic construct
ggggtcatacaggcttacc

SEQ ID NO: 12 - DNA, 20 bp, artificial sequence
  synthetic construct
tatgaaccttagcgcccaca

SEQ ID NO: 13 - DNA, 23 bp, artificial sequence
  synthetic construct
acttaatcaaactccgaatgtca

SEQ ID NO: 14 - DNA, 22 bp, artificial sequence
  synthetic construct
accaaacttaatcaaactccga

SEQ ID NO: 15 - DNA, 20 bp, artificial sequence
  synthetic construct
ggctgtttccctctcgacta

SEQ ID NO: 16 - DNA, 19 bp, artificial sequence
  synthetic construct
gacagactgtgggcgctaa

SEQ ID NO: 17 - DNA, 21 bp, artificial sequence
  synthetic construct
tcaataaagtatagcataaca

SEQ ID NO: 18 - DNA, 18 bp, artificial sequence
  synthetic construct
gacagactgtgggcgcta

SEQ ID NO: 19 - DNA, 21 bp, artificial sequence
  synthetic construct
ccaaacttaatcaaactccga

SEQ ID NO: 20 - DNA, 20 bp, artificial sequence
  synthetic construct
aacagacagactgtgggcgc

(19) United States
(12) Patent Application Publication
(10) Pub. No.: US 2021/0079453 A1
(43) Pub. Date: Mar. 18, 2021

(54) METHODS AND COMPOSITIONS FOR RICKETTSIACEAE DETECTION

(71) Applicant: The United States of America, as represented by the Secretary, Department of Health and Human Servic, Rockville, MD (US)

(72) Inventors: Cecilia Yukiko Kato, Atlanta, GA (US); Ida Ho Ting Chung, Atlanta, GA (US)

(21) Appl. No.: 16/970,318

(22) PCT Filed: Feb. 14, 2019
(86) PCT No.: PCT/US19/18039

§ 371 Date: Aug. 14, 2020

Related U.S. Application Data
(60) Provisional application No. 62/630,949, filed on Feb. 15, 2018.

(51) Int. Cl. C12Q 1/689; B01L 7/00

(52) U.S. Cl. CPC C12Q 1/689; C12Q 2600/16; C12Q 2600/158; B01L 7/525

(57) ABSTRACT

Primers, probes and kits for detection of Rickettsiaceare provided. Also provided are method and system for sensitive and specific detection of Rickettsiaceae using total nucleic acid isolated from a sample in a real-time reverse transcription PCR (Rrt-PCR), e.g. wherein 23S region is amplified and detected.

### PRIOR RELATED APPLICATION

The present application claims the benefit of priority of U.S. Provisional Application No. 62/630,949 filed Feb. 15, 2018, the content of which is incorporated herein by reference in its entirety.

### FIELD

The invention is related to compositions and method for the detection of Rickettsiaceae.

### BACKGROUND

Rickettsiaceae is family of Gram-negative obligate intracellular bacteria, currently classified as Alphaproteobacteria, which are the causative agents of rickettsioses. Rickettsiaceae family contains two genera, Rickettsia and Orientia. Some Rickettsiaceae bacteria are pathogenic to humans, causing various diseases ranging from mild to severe and/or fatal, such as typhus, spotted fevers, rickettsialpox and others. Low-prevalence of human rickettsioses and their often nonspecific symptoms, particularly at the early disease stages, make clinical diagnosis difficult. Timely diagnosis, however, is essential for accurate surveillance and effective treatment, especially in life-threatening rickettsioses. For example, Rocky Mountain spotted fever (RMSF) caused by Rickettsia ricketssii is a rapidly progressing disease treatable by the antibiotic doxycycline. The therapeutically effective window is quite narrow, and the delay in antibiotic administration correlates with unfavorable outcomes and death. In another example, scrub typhus, caused by Orientia tsutsugamushi, is widely prevalent but underdiagnosed. In 1997, the World Health Organization estimated the world prevalence at about 1 million cases of scrub typhus per year, with 1 billion people being at risk. Some sources report scrub typhus fatality rates of 1.4% in treated patients and 6% in untreated patients. Historically believed to be confined to Asia Pacific countries, recent reports documented scrub typhus establishment in the Arabian Peninsula, Chile and, possibly, Kenya. Underreporting of scrub typhus cases is believed to be due to nonspecific clinical presentation, misdiagnosis and lack of adequate diagnostic testing. Diagnostic assays allowing for early detection of Rickettsiaceae infections are crucial for timely treatment, collection of accurate surveillance information to generate reliable epidemiological data and for development of useful recommendations for treatment and prevention of rickettsioses throughout the world. Improved methods and reagents leading to sensitive and specific detection of human pathogenic Rickettsiaceae are necessary and valuable tools in research, clinical and epidemiological settings.

### SUMMARY

In order to facilitate diagnostics and surveillance of human rickettsioses, new and substantially improved PCR-based assays detecting Rickettsiaceae pathogens, including Rickettsia and Orientia, are described. The inventors have discovered PCR primers and probes that are useful for PCR-based detection of human pathogenic Rickettsiaceae with high specificity and sensitivity, as well as superior limit of detection (LOD). The primer and the probes discovered by the inventors are based on the sequences of 23S Rickettsiaceae ribosomal RNA (rRNA) of Rickettsia and Orientia and can be used in the detection methods that employ reverse transcriptase real-time polymerase chain reaction (rRT-PCR) techniques, which monitor the amplification of the nucleic acid sequences being detected in real time. The Rickettsiaceae assays employ a forward primer, a reverse primer and a probe for detection of a region of Rickettsiaceae 23S rRNA nucleic acid sequence.

Some of the described embodiments of the present invention relate to a new and substantially improved PCR-based assay detecting a number of Rickettsia species pathogenic to humans. The assay is referred to herein as the “RCKr assay.” As described below, the inventors have discovered PCR primers and probes that are useful for PCR-based detection of Rickettsia with high specificity and sensitivity, as well as superior limit of detection (LOD). The primer and the probes discovered by the inventors are based on the sequences of 23S Rickettsia ribosomal RNA (rRNA) and can be used in the detection methods that employ reverse transcriptase real-time polymerase chain reaction (rRT-PCR) techniques, which monitor the amplification of the nucleic acid sequences being detected in real time. RCKr assay employs a forward primer, a reverse primer and a probe for detection of a region of Rickettsia 23S rRNA nucleic acid sequence. The 23S rRNA sequence region being amplified and detected is highly conserved among the Rickettsia species that include human pathogens (“Rickettsia spp.”) and therefore serves as a specific marker for these Rickettsia species. Both genomic DNA encoding 23S rRNA and 23S rRNA are amplified and detected in RCKr assay. The assay generates a signal that can be interpreted to determine the presence or absence of a range of Rickettsia sequences in the sample. Rickettsia assays described in this document, including RCKr assay, use the primers and the probes also described in this document.

Some other of the described embodiments of the present invention relate to a new and substantially improved PCR-based assay detecting Orientia (for example, O. tsutsugamushi, a Rickettsiaceae bacterium that is an agent of scrub typhus). As described below, the inventors have discovered PCR primers and probes that are useful for PCR-based detection of Orientia with high specificity and sensitivity, as well as superior limit of detection (LOD). The primer and the probes discovered by the inventors are based on the sequences of 23S ribosomal RNA (rRNA) of Orientia tsutsugamushi and can be used in the detection methods that employ reverse transcriptase real-time polymerase chain reaction (rRT-PCR) techniques, which monitor the amplification of the nucleic acid sequences being detected in real time (rRT-PCR). Orientia assays use the primers and the probes described further in this document. The primers of the assay are used for the amplification of the 23S rRNA sequence region being detected, and the probe is used for detection of the amplification product. The assay generates a signal that can be interpreted to determine the presence or absence of Orientia sequences in the sample. Some embodiments of the Orientia assay can be referred to as the “OTSr assay.” OTSr assay employs a forward primer, a reverse primer and a probe for detection of a region of Orientia 23S rRNA nucleic acid sequence that serves as a specific marker for Orientia. Both genomic DNA encoding 23S rRNA and 23S rRNA are amplified and detected in OTSr assay.

Embodiments of the present invention can be used in clinical, research and public health fields. The primers and the probes discovered by the inventors can be combined in kits for conducting the assay. Accordingly, the present invention provides PCR primers, PCR probes, methods of using the PCR primers and/or probes, as well as the kits comprising the probes and/or primers. Some of the embodiments of the present invention are combination assays that can detect Rickettsia and Orientia and use at least some of the primers and/or probes of the Rickettsia assays, such as RCKr assay, and Orientia qassays, such as OTSr assay which can be combined in kits for conducting the combination assays.

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of some aspects of the invention and introduces some of the concepts that are further described in the “Description” section below. Some of the embodiments of the present invention are summarized below. This summary is not intended to identify key or essential features of the claimed subject matter, nor is it intended to be used in isolation to determine the scope of the claimed subject matter. The subject matter should be understood by reference to appropriate portions of the entire specification and each claim.

Some embodiments of the invention are methods of detecting presence or absence of Rickettsiaceae in a sample. Some other embodiments are methods of detecting in a sample a target nucleic acid sequence characteristic of Rickettsiaceae. The above methods may involve performing a real-time polymerase chain reaction (rPCR) on the sample or a nucleic acid isolated from the sample in order to detect a target nucleic acid sequence characteristic of Rickettsiaceae. In some embodiments, the methods are performed on a total nucleic acid (TNA) isolated from the sample. The methods can comprise isolating the total nucleic acid from the sample prior to performing rPCR. In some embodiments, rPCR is real-time reverse transcriptase PCR (rRT-PCR). In some of the above methods according to the embodiments of the present invention, both DNA and RNA comprising the target nucleic acid sequence is amplified during the rRT-PCR. The sample can be obtained from a patient suspected of being infected with Rickettsiaceae, such as Rickettsia, Orientia or both. The sample can be obtained from a patient suspected of having a rickettsiosis. The sample can be a tissue sample, a bodily fluid sample, a biological fluid sample, a blood sample, a serum sample, a plasma sample, a swab sample, or an isolate sample. The sample may have been previously stored. In the methods according to the embodiments of the present invention, the target nucleic acid sequence can be a sequence that is conserved across Rickettsia or Orientia species being detected. The above methods according to the embodiments of the present invention can detect Rickettsia and/or Orientia species pathogenic to humans. The target nucleic acid sequence can be a 23S rRNA nucleic acid sequence. In the methods according to the embodiments of the present invention, rPCR can be performed using at least one primer according to the embodiments of the present invention. Detection of the amplification product during rPCR can be performed using a probe according to embodiments of the present invention. Probes and primers according to the embodiments of the present invention are discussed elsewhere in this document.

Some other embodiments of the present invention are methods of assessing a Rickettsiaceae infection status of a patient. The Rickettsiaceae infection can be a Rickettsia infection or an Orientia infection. Such methods can involve detecting a target nucleic acid sequence characteristic of Rickettsiaceae in a sample according to the methods discussed above. They may involve performing a real-time polymerase chain reaction (rPCR) on the sample or a nucleic acid isolated from the sample in order to detect a target nucleic acid sequence characteristic of Rickettsiaceae. When the target nucleic acid sequence is detected, the status of the patient is infected with Rickettsiaceae, and when the target nucleic acid sequence is not detected, the status of the patient can be undetermined (meaning having no detectable Rickettsiaceae) or not infected with Rickettsiaceae. The methods of assessing Rickettsiaceae infection status of a patient may involve administering a treatment to the patient based on the assessed Rickettsiaceae infection status of the patient. In some embodiments, administering a treatment to the patient malso take into account clinical assessment of Rickettsiaceae infection status of the patient. Such methods may be referred to as methods assessing Rickettsia infection status of and treating a patient. The methods of assessing Rickettsiaceae infection status of a patient may also comprise a step of generating a report indicating the assessed Rickettsiaceae infection status of the patient. Such embodiments of the methods may be referred to as methods of generating reports.

Embodiments of the present invention include probes and primers based on Rickettsia sequences. For example, embodiments of the present invention include probes comprising an oligonucleotide comprising a sequence at least 90% identical to SEQ ID NO:3, SEQ ID NO:6 or SEQ ID NO:7, linked to at least one of a fluorophore moiety and a quencher moiety. The oligonucleotide can have a length of 25-35 bases. The sequence included in the nucleotide can be SEQ ID NO:3, SEQ ID NO:6 or SEQ ID NO:7. In some embodiments of the probes, the oligonucleotide is SEQ ID NO:3, SEQ ID NO:6 or SEQ ID NO:7 or a sequence at least 90% identical to SEQ ID NO:3, SEQ ID NO:6 or SEQ ID NO:7. In some embodiments of the probes, SEQ ID NO:3, SEQ ID NO:6 or SEQ ID NO:7 is linked to the fluorophore moiety and the quencher moiety. The fluorophore moiety used in the probes according to the embodiments of the present invention may include a fluorescein moiety. The fluorophore moiety used in the probes according to the embodiments of the present invention may be coupled to a 5′ terminus of the probe. The quencher moiety may be a BHQ quencher. The quencher moiety can be coupled to a 3′ terminus of the probe or to an internal base. In another example, embodiments of the present invention include primers that include a oligonucleotide having a sequence at least 90% identical to a SEQ ID NO:1, SEQ ID NO:2, SEQ ID NO:4 or SEQ ID NO:5. A primer according to the embodiments of the present invention can have a length of 20-30 bases. The oligonucleotide sequence can be SEQ ID NO:1, SEQ ID NO:2, SEQ ID NO:4 and SEQ ID NO:5. In some embodiments, the primer is SEQ ID NO:1, SEQ ID NO:2, SEQ ID NO:4 or SEQ ID NO:5 or a sequence at least 90% identical to a SEQ ID NO:1, SEQ ID NO:2, SEQ ID NO:4 or SEQ ID NO:5.

Embodiments of the present invention also include kits for detecting a Rickettsia nucleic acid sequence in a sample, comprising at least one probe according to the embodiments of the present invention and other reagents for performing an rPCR assays, such as an rRT-PCR assay. The other reagents may include at least one primer according to the embodiments of the present invention and comprising a sequence at least 90% identical to a sequence selected from the group consisting of SEQ ID NO:1, SEQ ID NO:2, SEQ ID NO:4, and SEQ ID NO:5. In one example, a kit includes a probe comprising the sequence at least 90% identical to SEQ ID NO:3 and at least one first primer comprising a sequence at least 90% identical to SEQ ID NO:1, SEQ ID NO:4 or SEQ ID NO:5 and a second primer comprising a sequence at least 90% identical to SEQ ID NO:2. In another example, a kit includes a probe comprising the sequence at least 90% identical to SEQ ID NO:6 and at least one primer selected from the group consisting of a first primer comprising a sequence at least 90% identical to SEQ ID NO:4 and a second primer comprising a sequence at least 90% identical to SEQ ID NO:2.

Embodiments of the present invention include probes and primers based on Orientia sequences. For example, embodiments of the present invention include probes comprising an oligonucleotide comprising a sequence at least 90% identical to SEQ ID NO:10, SEQ ID NO:13, SEQ ID NO:16, SEQ ID NO:17, SEQ ID NO:18, SEQ ID NO:19 or SEQ ID NO:20 linked to at least one of a fluorophore moiety and a quencher moiety. The oligonucleotide can have a length of 25-35 bases. The sequence included in the nucleotide can be SEQ ID NO:10, SEQ ID NO:13, SEQ ID NO:16, SEQ ID NO:17, SEQ ID NO:18, SEQ ID NO:19 or SEQ ID NO:20. In some embodiments of the probes, the oligonucleotide is SEQ ID NO:10, SEQ ID NO:13, SEQ ID NO:16, SEQ ID NO:17, SEQ ID NO:18, SEQ ID NO:19 or SEQ ID NO:20, or a sequence at least 90% identical to SEQ ID NO:10, SEQ ID NO:13, SEQ ID NO:16, SEQ ID NO:17, SEQ ID NO:18, SEQ ID NO:19 or SEQ ID NO:20. In some embodiments of the probes, SEQ ID NO:10, SEQ ID NO:13, SEQ ID NO:16, SEQ ID NO:17, SEQ ID NO:18, SEQ ID NO:19 or SEQ ID NO:20 is linked to the fluorophore moiety and the quencher moiety. The fluorophore moiety used in the probes according to the embodiments of the present invention may include a fluorescein moiety. The fluorophore moiety used in the probes according to the embodiments of the present invention may be coupled to a 5′ terminus of the probe. The quencher moiety may be a BHQ quencher. The quencher moiety can be coupled to a 3′ terminus of the probe or to an internal base. In another example, embodiments of the present invention include primers that include a oligonucleotide having a sequence at least 90% identical to a SEQ ID NO:8, SEQ ID NO:9, SEQ ID NO:11, SEQ ID NO:12, SEQ ID NO:14 or SEQ ID NO:15. A primer according to the embodiments of the present invention can have a length of 20-30 bases. The oligonucleotide sequence can be SEQ ID NO:8, SEQ ID NO:9, SEQ ID NO:11, SEQ ID NO:12, SEQ ID NO:14 or SEQ ID NO:15. In some embodiments, the primer is SEQ ID NO:8, SEQ ID NO:9, SEQ ID NO:11, SEQ ID NO:12, SEQ ID NO:14 or SEQ ID NO:15, or a sequence at least 90% identical to SEQ ID NO:8, SEQ ID NO:9, SEQ ID NO:11, SEQ ID NO:12, SEQ ID NO:14 or SEQ ID NO:15.

Embodiments of the present invention also include kits for detecting Orientia (such as Orientia tsutsugamushi) nucleic acid sequence in a sample, comprising at least one probe according to the embodiments of the present invention and other reagents for performing an rPCR assays, such as an rRT-PCR assay. The other reagents may include at least one primer according to the embodiments of the present invention and comprising a sequence at least 90% identical to SEQ ID NO:8, SEQ ID NO:9, SEQ ID NO:11, SEQ ID NO:12, SEQ ID NO:14 or SEQ ID NO:15, or a sequence selected from the group consisting of SEQ ID NO:8, SEQ ID NO:9, SEQ ID NO:11, SEQ ID NO:12, SEQ ID NO:14 and SEQ ID NO:15. In one example, a kit includes at least one probe comprising the sequence at least 90% identical to SEQ ID NO:10, SEQ ID NO:13, SEQ ID NO:16, SEQ ID NO:17, SEQ ID NO:18, SEQ ID NO:19 or SEQ ID NO:20, at least one first primer comprising a sequence at least 90% identical to SEQ ID NO:8, SEQ ID NO:11 or SEQ ID NO:14 and at least one second primer comprising a sequence at least 90% identical to SEQ ID NO:9, SEQ ID NO:12 or SEQ ID NO:15. In another example, a kit includes at least one probe comprising the sequence at least 90% identical to SEQ ID NO:10, at least one first primer comprising a sequence at least 90% identical to SEQ ID NO:8, and at least one second primer comprising a sequence at least 90% identical to SEQ ID NO:9. In one more example, a kit includes at least one probe comprising the sequence at least 90% identical to SEQ ID NO:10, SEQ ID NO:17, SEQ ID NO:18, SEQ ID NO:19 or SEQ ID NO:20, at least one first primer comprising a sequence at least 90% identical to SEQ ID NO:8, and at least one second primer comprising a sequence at least 90% identical to SEQ ID NO:9. In one more example, a kit includes at least one probe comprising the sequence at least 90% identical to SEQ ID NO:13, at least one first primer comprising a sequence at least 90% identical to SEQ ID NO:11, and at least one second primer comprising a sequence at least 90% identical to SEQ ID NO:12. In one more example, a kit includes at least one probe comprising the sequence at least 90% identical to SEQ ID NO:16, at least one first primer comprising a sequence at least 90% identical to SEQ ID NO:14, and at least one second primer comprising a sequence at least 90% identical to SEQ ID NO:15.

Embodiments of the present invention also include kits for detecting Rickettsia and Orientia (such as Orientia tsutsugamushi) nucleic acid sequences in a sample. The kits can comprise at least one Rickettsia assay probe according to the embodiments of the present invention, at least one Orientia assay probe according to the embodiments of the present invention, and can further comprise other reagents for performing rPCR assays, such as an rRT-PCR assay. The other reagents may include one or more primers according to the embodiments of the present invention that are useful for performing Rickettsia assays and/or Orientia assays. The kits according to the embodiments of the present invention may further include one or more reagents for purifying total nucleic acid from the sample. The kits according to the embodiments of the present invention may also include one or more devices for purifying total nucleic acid from the sample. Embodiments of the present invention also include kits for amplifying a nucleic acid sequence of a region of Rickettsiaceae 23S rRNA nucleic acid sequence, comprising at least one primer according to the embodiments of the present invention and one or more other ingredients for performing a PCR.

Methods of using the above primers, probes and/or kits are also included among the embodiments of the present invention. One example is a method of amplifying a nucleic acid sequence that involves contacting the sample with at least one primer according to the embodiments of the present invention and performing a PCR. Another example is a method of detecting a nucleic acid comprising a region of Rickettsiaceae 23S rRNA nucleic acid sequence in the sample that involves performing the above method of amplifying and detecting one or more products of the amplification. The nucleic acid being detected is present in the sample if the one or more products of the amplification are detected.

Also among the embodiments of the present invention are systems for detecting presence or absence of Rickettsiaceae in a sample. Such systems include one or more stations. For example, they may optionally include a station for isolating the total nucleic acid from the sample. A system may include a station for performing rPCR, such as rRT-PCR, on the isolated total nucleic acid to detect a target nucleic acid sequence characteristic of Rickettsiaceae. A system may include an optional a station for generating a report on the presence or absence of Rickettsiaceae in the sample based on detected presence or absence of the target nucleic acid sequence. The system may include a computer and various components thereof (processor, memory, computer code, etc.).

Other objects and advantages of the invention will be apparent from the following description of embodiments of the invention.

### BRIEF DESCRIPTION OF THE FIGURES

FIGS. 1A, 1B, 1C and 1D show the examples of the chemical structures of Black Hole Quencher® dyes (Biosearch Technologies, Petaluma, Calif.).

FIG. 2 is a schematic illustration of TaqMan probe.

FIG. 3 is a schematic illustration of Zen probe.

FIG. 4 shows chemical structures of pdU-CE Phosphoramidite (5′-Dimethoxytrityl-5-(1-Propynyl)-2′-deoxyUridine,3′-[(2-cyanoethyl)-(N,N-diisopropyl)]-phosphoramidite) and pdC-CE Phosphoramidite (5′-Dimethoxytrityl-N4-diisobutylaminomethylidene-5-(1-Propynyl)-2′-deoxyCytidine,3′-[(2-cyanoethyl)-(N,N-diisopropyl)]-phosphoramidite).

FIG. 5 is a schematic of a system according to some embodiments of the invention.

FIG. 6 is a bar chart illustrating analytical sensitivity of RCKr assay.

FIG. 7 is a bar chart illustrating analytical sensitivity of RCKr assay.

FIG. 8 is a dot plot illustrating the results of comparative accuracy testing of RCKr assay using RCKr and PanR8 assays performed on total nucleic acids extracted from a contrived and blinded panel of samples.

FIG. 9 is a bar chart illustrating the results of the RCKr assay performed on two different instruments with two different master mix reagents.

FIG. 10 is a dot plot illustrating analytical sensitivity of OTSr assay performed on Orientia tsutsugamushi samples of known concentrations.

FIG. 11 is a bar graph illustrating comparative performance of Ori5 and OTSr assays on two different instruments with two different master mix reagents.

### DESCRIPTION

The present description describes and refers to various embodiments of the invention. No particular embodiment is intended to define the scope of the invention. Rather, the embodiments merely provide non-limiting examples of various methods, compositions, kits, systems etc. that are at least included within the scope of the invention. A number of terms and concepts are discussed in the present description, which are intended to aid the understanding of various embodiments of the invention in conjunction with the rest of the present document.

Among other things, the present document describes improved real-time polymerase chain reaction (rPCR) assays useful for sensitive detection of Rickettsiaceae bacteria, which are Gram-negative obligate intracellular organisms currently classified as Alphaproteobacteria. Some of Rickettsiaceae are causative agents of rickettsioses in humans and animals throughout the world. Rickettsiaceae family includes two orders, Rickettsia and Orientia. The present document describes improved rPCR assays useful for sensitive detection of a range of Rickettsia species including those pathogenic to humans. Such assays can be referred to as “Rickettsia assays.” The present document also describes improved rPCR assays useful for detection of Orientia, such as Orientia tsutsugamushi, a causative agent of scrub typhus.

TABLE 1
Rickettsial disease

| | | | |
|---|---|---|---|
| DISEASE | SPECIES | VECTOR | GEOGRAPHIC DISTRIBUTION |

Spotted Fever (SF) antigenic group

| | | | |
|---|---|---|---|
| Rickettsiosis | Rickettsia | Tick | South Africa, Morocco, |
| (unspecified) | aeschlimannii | | Mediterranean, Central and |
| | | | Southern Europe |
| African tick-bite fever | R. africae | Tick | Sub-Saharan Africa, West Indies |
| Rickettsialpox | R. akari | Mite | Countries of the former Soviet |
| | | | Union, South Africa, Korea, |
| | | | Turkey, Balkan countries, North |
| | | | and South America |
| Queensland tick | R. australis | Tick | Australia, Tasmania |
| typhus | | | |
| Mediterranean SF, | R. conorii | Tick | Southern Europe, south and west |
| Boutonneuse fever | | | Asia, Africa, India |
| Cat flea typhus, flea- | R. felis | Flea | Europe, North and South America, |
| borne SF | | | Africa, Asia |
| Far Eastern SF | R. | Tick | Far East of Russia, Northern China, |
| | heilongjiangensis | | eastern Asia |
| Aneruptive fever | R. helvetica | Tick | Central and Northern Europe, Asia |
| Flinders Island SF | R. honei | Tick | Australia, Thailand |
| Japanese SF | R. japonica | Tick | Japan |
| Mediterranean SF-like | R. massiliae | Tick | Europe, Central Africa, and Mali |
| disease | | | |
| Mediterranean SF-like | R. monacensis | Tick | Europe, North Africa |
| illness | | | |
| Rickettsia parkeri | R. parkeri | Tick | North and South America |
| rickettsiosis, | | | |
| Maculatum infection | | | |
| Tickborne | R. raoultii | Tick | Europe, Asia |
| lymphadenopathy | | | |
| Rocky Mountain SF | R. rickettsii | Tick | North, Central, and South America |
| North Asian tick | R. sibirica | Tick | Russia, China, Mongolia |
| typhus, Siberian tick | | | |
| typhus | | | |
| Lymphangitis- | R. sibirica | Tick | Southern Europe, Portugal, China, |
| associated rickettsiosis | mongolotimonae | | Africa |
| Tickborne | R. slovaca | Tick | Southern and Eastern Europe, Asia |
| lymphadenopathy, | | | |
| Dermacentor-borne | | | |
| necrosis and | | | |
| lymphadenopathy | | | |

Typhus Fever Antigenic group

| | | | |
|---|---|---|---|
| Epidemic North Asian | R. prowazekii | Lice, tick, | Central Africa, Europe, Asia, |
| tick typhus, sylvatic | | flying | Central, North and South America |
| North Asian tick | | squirrel, | |
| typhus | | flea | |
| Murine North Asian | R. typhi | Flea | Tropical and subtropical areas |
| tick typhus | | | worldwide |

Scrub Typhus

| | | | |
|---|---|---|---|
| Scrub typhus | Orienta | Larval | Siberia, Southeast Asia, Indonesia, |
| | tsutsugamushi | mites | China, Japan, India, Pakistan, |
| | | | Afghanistan, Northern Australia |
| Scrub typhus | O. chuto | Larval | Arabian peninsula, Kenya, West |
| | | mites | Africa |

Some of pathogenic Rickettsiaceae are shown in Table 1. Pathogenic Rickettsia include, but are not limited to, R. aeschlimannii, R. africae, R. akari, R. australis R. conorii, R. felis, R. heilongjiangensis, R. helvetica, R. honei, R. japonica, R. massiliae, R. monacensis, R. parkeri, R. raoultii, R. rickettsia, R. sibirica, R. sibirica mongolotimonae, R. slovaca, R. prowazekii and R. typhi. Rocky Mountain Spotted Fever (RMSF) was the first rickettsial disease described in 1899. Rickettsia rickettsii is the etiological agent of RMSF and causes an acute infection with fever, headache and malaise. The disease may rapidly progress to involve multi-organ failure and death. While it is treatable by the administration of the tetracycline antibiotic, doxycycline, early treatment administration is crucial. RMSF is 20-25% fatal if untreated and in 5% of treated cases.

Traditionally classified according to their antigenic properties, two main Rickettsia groups are associated with diseases pathogenic to humans: spotted fever group Rickettsia (SFGR) including RMSF, African tick bite fever, Mediterranean spotted fever, and rickettsial pox; and typhus group Rickettsia (TGR) which include epidemic typhus and murine typhus. The predominant symptoms of these diseases include headache, fever, and malaise, with or without the observation of rash or specific skin lesions (eschars). The nonspecific symptoms of these low prevalence diseases at the early stage of acute illness make their diagnosis difficult, so cases are often missed even in endemic areas. The accepted gold standard for diagnostic detection of Rickettsia in clinical settings is the observation of a four-fold rise (by indirect fluorescent antibody assay (IFA) in patient antibody titers to specific rickettsial antigens of paired acute and convalescent samples taken at least three to six weeks apart. However, the antibody titers become detectable 2-4 weeks after the resolution of symptoms. Thus, serologic detection is unhelpful as an early diagnostic tool because of the late presentation of reliable antibody titers. Serology can only be used to confirm a diagnosis after recovery. Cell culture isolation from patient samples is another gold standard for Rickettsia infection confirmation, but takes a long time and is also unsuitable for early diagnostics. In addition, it is sensitive to antibiotic treatment before sample draw. Acute stage testing can be performed by immunohistochemistry of biopsied tissue, which is a laborious and sampling is an invasive process.

Pathogenic Orientia include, but are not limited two, two currently known species, O. tsutsugamushi and O. chuto, both of which are causative agents of scrub typhus, a life-threatening human disease. Small rodents are believed to serve as animal reservoirs of Orientia. Scrub typhus is spread to people through bites of infected chiggers (larval mites). Symptoms of scrub typhus usually begin within ten days of being bitten. Signs and symptoms may include fever and chills, headache, body aches and muscle pain, a dark, scab-like region at the site of the chigger bite (known as eschar), mental changes ranging from confusion to coma, enlarged lymph nodes, rash, etc. Infected individuals with severe illness may develop organ failure and bleeding, which can be fatal. Early symptoms of scrub typhus are unspecific and similar to those exhibited in other infectious diseases, such as leptospirosis, murine typhus, malaria and dengue fever. Although scrub typhus is easily treatable with doxycycline if diagnosed early, in the absence of appropriately early treatment the fatality rate can be relatively high, reported by some sources at about 6% and up to 40-50% in untreated patients. Orientia is known to be resistant to many common classes of antibiotics, including ß-lactams, fluoroquinolones, and aminoglycosides, which makes the early diagnosis particularly important for timely determination and administration of appropriate treatment. The majority of currently available scrub typhus diagnostic tests are serological, including those based on indirect immunofluorescence antibody assay, dot-blot analysis, ELISA and passive hemagglutination assay. Molecular diagnostic testing for scrub typhus is limited to certain reference laboratories, state laboratories, research institutions and only a small number of commercial laboratories, largely because the limit of detection (LOD) of currently available molecular assays using DNA detection is not adequate for reproducibly detecting the low levels of Orientia circulating in blood.

The ability to detect Rickettsiaceae (including Rickettsia and Orientia) reliably at low levels is essential for early diagnosis effective treatment, especially in the cases of human infections, such as R. rickettsii infection (RMSF) and Orientia infection (scrub typhus), in which a delay in doxycycline administration leads to unfavorable outcomes and death. Recently, molecular detection by PCR-based methods has become more widely used for the confirmation of acute stage rickettsial infections in patient samples. As an obligate intracellular organism, Rickettsiaceae infect the host's nucleated cells where they multiply and the organisms spread cell to cell in the host's tissue. In vertebrate animals, Rickettsiaceae enter the blood and circulates through body. Rickettsiaceae detection can therefore be performed on a variety of biological samples. While obtaining blood and serum samples is easy and convenient, the testing of the blood and serum samples for the purpose of detecting Rickettsiaceae is challenging due to low titers of bacteria present. Because Rickettsiaceae mainly infect nucleated cells, the levels of Rickettsiaceae bacteria in blood are generally low, except for in advanced disease state. Even at the lower limits of detection sensitivity for highly sensitive PCR-based assays, such as rPCR, an efficiently repeatable assay at ˜9 copies per reaction (5 μL of sample) currently still requires approximately 1,800 genome copies of the organism to be present in 1 mL of blood.

Low analytical sensitivity of currently available rPCR assays is particularly troubling in the case of RMSF, because of the rapid progression of this illness and the fact that diagnosis is missed in 60-85% of cases on the initial evaluation. Clinical diagnosis of the disease is difficult because RMSF is rare, and the early stages of the illness resemble other less severe infections. Proper antibiotic therapy must be administered within the first 5 days of symptoms or there is higher chance of a poor outcome or death. The diagnostic and therapeutic window for best outcome is narrow; most deaths occur within 7-9 days after onset. In the case of scrub typhus, lack of treatment leads to significantly increased patient mortality. Furthermore, scrub typhus surveillance is impeded by nonspecific clinical presentation, misdiagnosis and lack of appropriately sensitive diagnostic tests. Accordingly, PCR-based assays with high analytical sensitivity capable of detecting low levels of Rickettsiaceae bacteria, such as the low levels found in patient's blood or serum at the early stages of Rickettsia and Orientia infections, is highly useful and advantageous for early diagnosis of rickettsioses. Such assays are described herein.

In contrast to the currently available PCR-based assays for Rickettsiaceae detection, which target single copy genes in the DNA of the rickettsial genome, the improved rPCR assays described in this document are reverse transcriptase real-time PCR (rRT-PCR) assays, which amplify target nucleic acid sequences of both DNA and RNA. The assays use both reverse transcriptase and DNA polymerase to amplify target nucleic acid sequences in total nucleic acid isolated from the patient samples. One embodiment of the improved rPCR assay is a Rickettsia assay that amplifies and detects a target sequence within a conserved region of 23S rRNA sequence, which allows for the detection of a wide range of Rickettsia species include those pathogenic to humans, and therefore can be referred to as “pan-Rickettsia assay.” The primers used in the pan-Rickettsia assays according to the embodiments of the present invention are useful for amplification of a region of Rickettsia 23S ribosomal RNA (rRNA), which can be referred to as “23 S rRNA target region.” The 23S rRNA target region is a part of the operon that includes the methionyl-tRNA_{f}^{Met} formyltransferase (fmt), 23S rRNA, 5S rRNA gene cluster, which is well conserved among the Rickettsia that are known to be pathogenic to humans. This gene arrangement is unlike the 16S (rrs), 23S, and 5S rRNA operon typically observed in other bacteria and is currently thought to have occurred before the Rickettsia spp. and Orientia spp. divide. The conserved sequences flanking the 23S rRNA and 5S rRNA intergenic region are therefore useful as a target for Rickettsia detection generally, while the sequence variation in the intergenic region is useful for species differentiation. Another embodiment of the improved rPCR assay is an Orientia assay that amplifies and detects a target sequence within a conserved region of Orientia 23S rRNA sequence. The Orientia genome has a high degree of plasticity, which poses a diagnostic challenge. The improved rPCR assay described in this document allows for the detection of a wide range of Orientia strains. The probes used in the improved rPCR assays are used for specific detection of the amplification products produced in the course of amplifying the 23S rRNA target region or a region of 23S rRNA comprising the sequence to which the probe sequence can hybridize. For example, when contacted with the sample in the context of an rRT-PCR assay according to the embodiments of the present invention, a reverse transcriptase reaction transcribes the rRNA sequence into DNA. The newly transcribed DNA and the rDNA sequences are then available as template for detection by PCR amplification. The primers amplify a 23S rRNA target region from genomic DNA and/or rRNA, and the probes target the sequence within the amplified target region, generating a signal that can be interpreted to determine the presence or absence of target sequences in the sample.

The following abbreviations may be used, among others, in the present document: PCR—polymerase chain reaction; RMSF—Rocky Mountain spotted fever; RT—reverse transcriptase; RT-PCR—reverse transcriptase PCR; rRT-PCR—real-time RT-PCR; rPCR—real-time PCR; RNA—ribonucleic acid; DNA—deoxyribonucleic acid; DPI₃-dihydrocyclopyrroloindole tripeptide; TNA—total nucleic acid; BHQ-Black Hole Quencher, FAM—6 carboxyfluorescein; FRET—fluorescence resonance energy transfer; LOD—limit of detection; MGB—minor groove binder; TET—tetrachlorofluorescein, HEX—hexachloro-6-carboxyflourescein; pdC—C-5 propynyl-dC; pdU—C-5 propynyl-dU, MGP—magnetic glass particle, PPV—positive predictive value, NPV—negative predictive value.

The term “amplification” and the related terms are used to refer to the process or to the result of the process used to increase the number of copies of a nucleic acid molecule. The resulting products can be called “amplification products” or “amplicons.” An example of an amplification technique is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify a number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

The term “assay” and the related terms are used to broadly refer to methods, processes or procedures used for assessing or measuring the presence, absence or amount or the of a target entity (the analyte). The assays according to the embodiments of the present invention are used to assess the presence, absence or amount of Rickettsiaceae bacteria in a sample.

The terms “assess,” “assessment,” “assessing” and related terms are used in reference to Rickettsiaceae bacteria and their genes and/or nucleic acid sequences to describe inferring the presence, the absence or the amount of Rickettsiaceae a sample based on the detected presence, absence or amount of Rickettsiaceae sequences.

The terms “to contact,” “contacting” and the related terms can be used to describe the process or the result of placing chemical compounds in the same reaction environment, such as the same reaction vessel or solution.

The terms “detect,” “detecting,” “detection” and similar terms are used in this document to broadly to refer to a process of discovering or determining the presence or an absence, as well as a degree, quantity, or level, or probability of occurrence of something. The terms necessarily involve a physical transformation of matter, such as nucleic acid amplification by PCR. For example, the term “detecting” when used in the context of Rickettsiaceae detection, can denote discovery or determination of the presence, absence, level or quantity, as well as a probability or likelihood of the presence or absence of Rickettsiaceae bacteria being detected. It is to be understood that the expressions “detecting the presence or absence,” “detection of the presence or absence” and related expressions, include qualitative, semi-quantitative and quantitative detection. Quantitative detection includes a determination of the level, quantity or amounts of the analyte being detected, such as a Rickettsia or Orientia target sequence, in the sample, on which the detection process is performed with standards for reference. Semi-quantitative detection and qualitative detection include inferring the presence or absence of the analyte in a sample based on a detection parameter being above or below a predetermined value.

The terms “detection limit,” “limit of detection,” abbreviation “LOD” and other related terms can be used in the context of the embodiments of the present invention to refer to the lowest analyte concentration or amount that can be reliably (for example, reproducibly) detected for a given type of sample and/or assay. Limit of detection can be determined by testing serial dilutions of a sample known to contain the analyte and determining the lowest dilution at which detection occurs. The limit of detection of the assays described in this document can be expressed as concentration, such as target nucleic acid copy number or RNA copy number per volume, or amount, such as the number of copies of a particular sequence that can be detected.

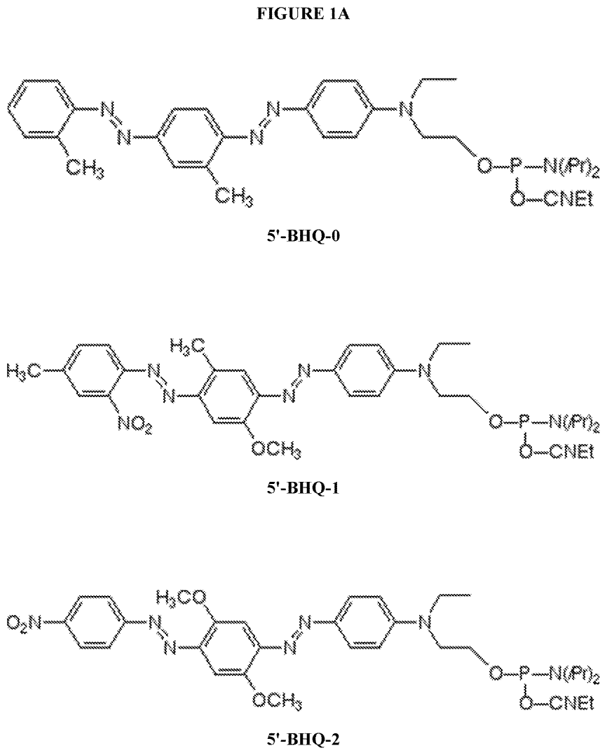

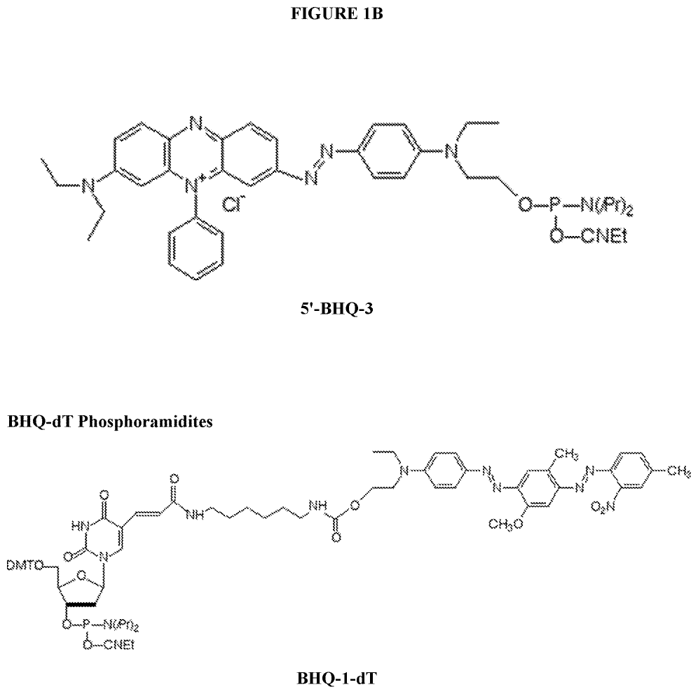

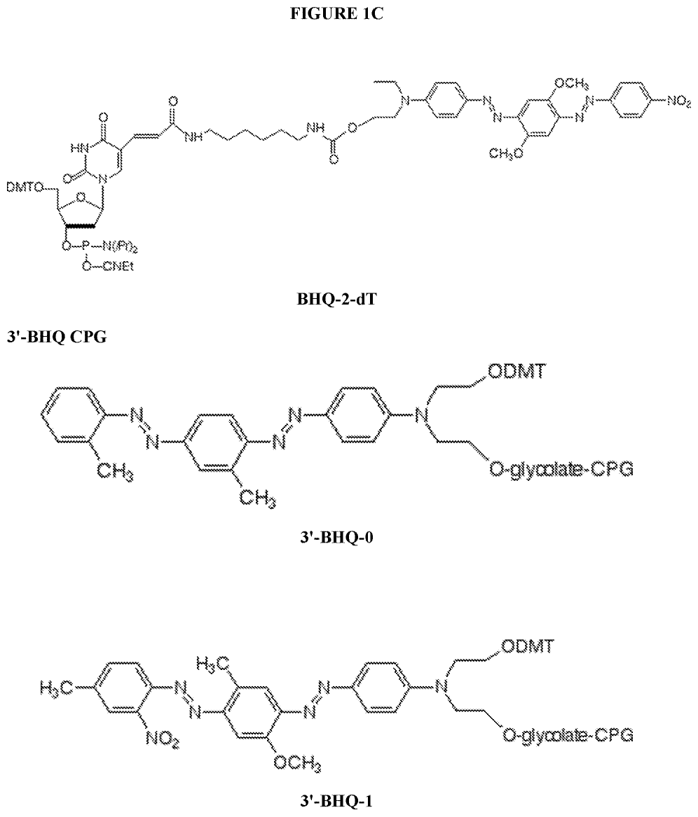

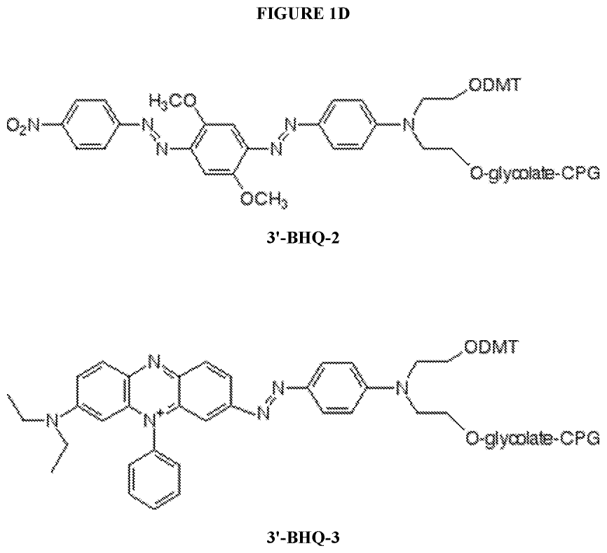

The term “fluorescence” broadly refers to the process or the result of the emission of light by a substance that has absorbed light or other electromagnetic radiation. The following terms and concepts can be used to describe how fluorescence is employed in the embodiments of the present invention. Fluorophores or fluorescent dyes are chemical compounds or moieties that can re-emit light upon light excitation. Fluorophores typically contain several combined aromatic groups, or plane or cyclic molecules with several it bonds. A fluorophore absorbs light energy of a specific wavelength and re-emits light at a longer wavelength. When a fluorophore is excited at a particular wavelength, it is promoted to an excited state. In the absence of a quencher, the excited dye emits light in returning to the ground state. When a quencher is present in physical proximity, the excited fluorophore can return to the ground state by transferring its energy to the quencher, without the emission of light. Different types of quenchers exist. One quenching mechanism relies on the ability of the fluorophore to transfer energy to a second fluorophore by fluorescence resonance energy transfer (FRET). This returns the fluorophore to the ground state and generates the quencher excited state. The quencher then returns to the ground state through emissive decay (fluorescence). In order for this to happen, the emission spectrum of the fluorophore must overlap with the absorption spectrum of the second fluorophore (quencher). One example of such the fluorophore/quencher pair is fluorescein (used as the fluorescent reporter dye) and rhodamine as the quencher (FAM/TAM probes). However, quencher fluorescence can increase background noise due to overlap between the quencher and reporter fluorescence spectra. Dark quenchers are dyes with no native fluorescence. Dark quenchers return from the excited state to the ground state via non-radiative decay pathways, without the emission of light. In dark decay, energy is given off via molecular vibrations (heat). With the typical concentration of probe being in μM range or less, the heat from radiationless decay is too small to affect the temperature of the solution. Thus, the term “dark quencher” can be used in the context of the present invention to refer to a substance or moiety that absorbs excitation energy from a fluorophore and dissipates the energy as heat; while the term “fluorescent quencher” can be used to refer to a substance or moiety that re-emits much of this energy as light. Dark quenchers do not occupy an emission bandwidth and allow multiplexing, when two or more reporter-quencher probes are used together. BHQ quenchers, some of which are illustrated in FIG. 1, are examples of dark quenchers.

The term “isolated” can be used in this document to refer to a biological component (such as a nucleic acid) that has been substantially separated or purified away from other biological components (such as cell debris, or other proteins). Biological components that have been “isolated” include those components purified by standard purification methods. The term also embraces recombinant nucleic acids and viruses, as well as chemically synthesized nucleic acids.

“Moiety” refers to a part or functional group of a molecule.

“Oligonucleotide” and related terms are used in this document to refer to nucleic acid molecules, such as RNA or DNA molecules or their modifications, 200 bases long or less. The term “oligonucleotide” includes naturally occurring or non-natural (synthetic) nucleic acid sequences, as well as sequences containing residues, liners, labels etc. that do not naturally occur in nucleic acids, including modified natural nucleotides, etc.

“Primers” (singular—“primer”) are strands of short nucleic acid sequences, such as a DNA oligonucleotides, used as starting points for DNA synthesis during nucleic acid amplification reaction, such as PCR. Primers contain oligonucleotides with a sequences that can be annealed to a complementary target nucleic acid molecule by nucleic acid hybridization to form a hybrid between the primer and the target nucleic acid strand. A primer can be described as “specific” for a target nucleic acid. During the amplification reaction, a primer can be extended along the target nucleic acid molecule by a polymerase enzyme. Thus, primers can be used to amplify a target nucleic acid molecule (such as a portion of Rickettsiaceae bacteria nucleic acid), wherein the sequence of the primer is specific for the target nucleic acid molecule, for example so that the primer will hybridize to the target nucleic acid molecule under high or very high stringency hybridization conditions employed in some parts of the PCR cycle. Primers are often characterized by “Primer Melting Temperature” (Tₘ), which is the temperature at which one half of the DNA duplex will dissociate to become single stranded and indicates the duplex stability. Primer melting temperature depends, in part, on its length and nucleotide sequence. A primer according to the embodiments of the present invention can be is at least 15 nucleotides in length, such as at least 15 contiguous nucleotides complementary to a target nucleic acid molecule, including the primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or 50 or more contiguous nucleotides complementary to the target nucleic acid molecule to be amplified, such as a primer of 15-60 nucleotides, 15-50 nucleotides, 20-40 nucleotides, or 15-30 nucleotides. Primers are generally used in pairs for amplification of a nucleic acid sequence, for example, by PCR, real-time PCR, or other nucleic-acid amplification methods. An “upstream” or “forward” primer is a primer 5′ to a reference point on a nucleic acid sequence. A “downstream” or “reverse” primer is a primer 3′ to a reference point on a nucleic acid sequence. At least one forward and one reverse primer are included in an amplification reaction. Primers can contain one or more detectable labels or reporters, meaning moieties that are detectable by various methods or assist in detection. One example of a detectable label or reporters is a fluorescent dye, such as WellRed fluorescent dyes (supplied by Beckman Coulter., Inc.). Another example is biotin. Biotinylated primers can be used, for example, in Luminex technology and Pyrosequencing techniques. Biotin can be added to oligonucleotides on either terminus (“standard” biotin), as well as internally through a modified thymidine residue (biotin-dT). In some cases, primers act as probes during detection. For example, so-called scorpion primers can be used for detection in real-time PCR assays. Scorpion primers contain a stem-and-loop oligonucleotide structure with a 5′ fluorescent report and a 3′ quencher (“probe sequence”), which is attached to 5′ terminus of the oligonucleotide specific for the target nucleic acid sequence. During the annealing phase of the PCR, the probe sequence hybridizes to the newly formed complementary target sequence, separating the fluorophore and the quencher dyes and leading to emission of fluorescence signal.

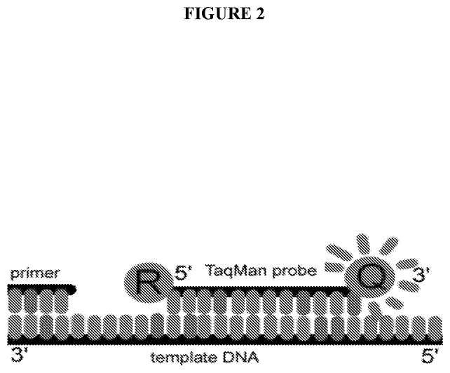

The term “probe” (plural—“probes”) and related terms are used in this document to refer to a molecule containing an oligonucleotide of variable length that is capable of hybridizing to a target nucleic acid sequence. The probe can be described as “specific for” the target nucleic acid sequence. Probes can be characterized by their Tₘ. The probes according to the embodiments of the present invention include rRT-PCR probes, which are probes capable of hybridizing to rRT-PCR amplification products. A probe can contain one or more detectable labels or reporters, meaning moieties that are detectable by various methods or assist in detection. For example, a variation of the probes described in this document are fluorescent reporter probes useful in rRT-PCR assays. One example of such probes are the so-called hydrolysis probes, such TaqMan® probes. TaqMan® probes are oligonucleotide probes that contain a fluorescence reporter moiety covalently attached to the 5′ end and a quencher moiety, which can be attached at the 3′ end or at an internal nucleotide, which reduces the fluorescence emitted by the fluorescent reporter. FIG. 2 schematically illustrates a TaqMan® probe (R denotes a reporter; Q denotes a quencher). Some examples of fluorophores-suitable for use as fluorescent reporter dyes in TaqMan® probes are 6-carboxyfluorescein (FAM), tetrachlorofluorescein (TET), hexachloro-6-carboxyflourescein (HEX). When a probe is intact, the quencher suppresses the fluorescence of the fluorescence reporter dye. When the probe is used in real-time PCR, during the extension phase, the probe is cleaved by the exonuclease activity of the DNA polymerase, releasing the fluorophore. The fluorophore release results in an increase in fluorescence signal, which is proportionate to the amount of the PCR product.

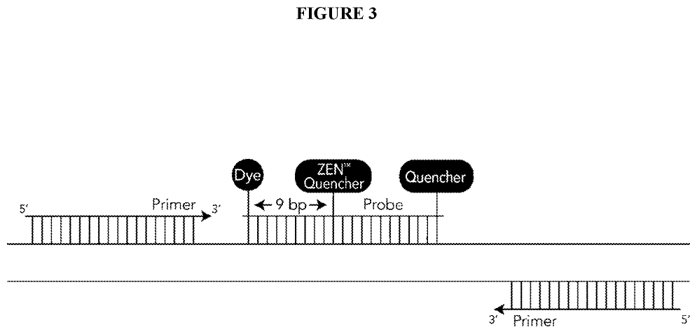

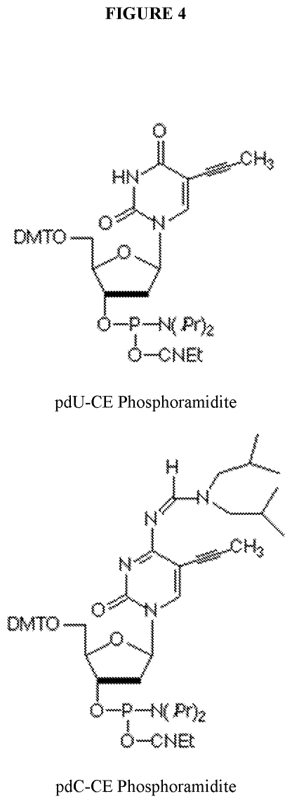

Variations and modifications of hydrolysis probes are possible. One example of such a modification is incorporation-of conjugated Minor Groove Binder (MGB) groups into a probe. The MGB groups act as duplex stabilizers. MGB probes typically incorporate a 5′ reporter dye and a 3′ nonfluorescent quencher, with the MGB moiety attached to the quencher molecule. One example of an MGB moiety is dihydrocyclopyrroloindole tripeptide (DPI₃), which folds into the minor groove formed by the terminal 5-6 bp of the probe. Such probes form extremely stable duplexes with single-stranded DNA targets, allowing shorter probes to be used. In comparison with unmodified DNA, MGB probes have higher melting temperature (Tₘ) and increased specificity. Another example is incorporation of modified bases, such as propyne derivatives, into nucleotides. For example, substitution of C-5 propynyl-dC (pdC) for dC and C-5 propynyl-dU (pdU) for dT (both illustrated in FIG. 4) are effective strategies for enhancing base pairing. These base substitutions enhance duplex stability and increase probe Tₘ by the following amounts: C-5 propynyl-C—2.8° C. per substitution; C-5 propynyl-U—1.7° C. per substitution. So-called BHQplus® probes provided by Biosearch technologies employ pdC and pdU substitutions in combination with BHQ dark quenchers. BHQplus and MGB probes can be used with oligonucleotides of shorter length and thus achieve an enhanced target specificity Another example of the probes used in real-time PCR assays are dual hybridization probes, which employ fluorescence resonance energy transfer (FRET) between the fluorophores on two different probes. Two fluorophore-labeled sequence-specific probes are designed to bind to the PCR product during the annealing phase of PCR, which results in an energy transfer from a donor fluorophore to an acceptor fluorophore. This results in an increase in fluorescence during the annealing phase. Some other examples of suitable probes are ZEN® Double-Quenched Probes (manufactured by Integrated DNA Technologies, Coraville, Iowa) (illustrated in FIG. 3) and QSY® probe from ThermoFisher Scientific, Waltham, Mass.

The terms “sample” or “samples,” as used interchangeably herein, include samples originating from human or animal subject (such as, but not limited to, samples of human or animal cells, tissues or bodily fluids and excretions) as well as samples prepared or generated by various laboratory and industrial processes. For example, a sample can be a biological sample, biopsy sample, autopsy sample, tissue sample, organ sample (for example, but not limited to blood, spleen, kidney, ling, liver, skin, nerve tissue or brain sample), bodily fluid (blood, serum, plasma, sperm, cerebrospinal fluid, urine etc.) sample, biological fluid (bodily fluid, cell or tissue culture, lysate or extract of a biological sample of any origin etc.) sample, cell sample, blood sample, serum sample, plasma sample, etc. A sample can be a fluid or tissue swab (“swab sample”), a preserved tissue, blood or sell sample (such as slide or a block), etc. A sample can be a sample of an isolated nucleic acid, such as DNA, RNA or TNA. A sample can be directly obtained from a human or animal organism, obtained from the environment (such as food samples, water samples, surface swabs) propagated, cultured, synthesized or otherwise artificially produced. For example, a sample can be an infectious organism isolate, including a primary isolate from a sample obtained from an infected individual, or an isolate propagated in the laboratory or industrially using various techniques, including recombinant techniques, tissue culture or propagation in eggs, plants or nonhuman animals at various stages of development. Samples can be subject to various purification, storage or processing procedures before being analyzed according to the methods described herein. Samples can also be obtained at various steps of laboratory reactions and assays. For example, a sample may be produced by a PCR reaction. The methods described in this document may involve several different samples at different steps of the method. For example, a first sample may subjected to a PCR amplification step, and a second sample may be obtained after a PCR amplification step and subsequently processed or analyzed in a subsequent detection or analysis step or steps. Generally, the terms “sample” or “samples” are not intended to be limited by their source, origin, manner of procurement, treatment, processing, storage or analysis, or any modification.

In the context of the present invention, a sample may be a sample of a nucleic acid, such as DNA and/or RNA. A sample may be a so-called “total nucleic acid” (TNA) sample, meaning a sample of both RNA and DNA isolated from a biological material. TNA samples can be be prepared by various methods that allow for isolation of DNA and RNA from biological materials. For example, DNA and/or RNA samples can be prepared using magnetic bead based or spin column based extractions. The isolation steps include the addition of lysis buffer/proteinase cocktail to each sample, followed by an inactivation step. The mixture can be added to Magnetic Glass Particles (MGPs), where DNA/RNA binds to and purified through wash steps. Alternatively, the mixture can be placed into a spin column where DNA/RNA will bind and purified through wash steps. The total extracted product is recovered in a small volume of elution buffer. TNA samples can also prepared by DNA extraction methods that do not involve RNA digestion (RNase treatment) or by RNA extraction methods that do not involve DNA digestion (DNase treatment).

The term “region” can be used in this document to refer to a part of a nucleic acid sequence, a part of a gene, or a part of a segment of Rickettsiaceae nucleic acid, and can be used interchangeably with the term “sequence.” For example, a region may be a region of Rickettsiaceae 23 S rRNA nucleic acid sequence, such as a Rickettsia or Orientia 23S rRNA nucleic acid sequence.

The terms “sensitivity” and “specificity” can be used to refer to statistical measures of the performance of assays and methods described in this document. Diagnostic sensitivity refers to a proportion of positive results which are correctly identified by a test. Diagnostic specificity measures a proportion of the negative results that are correctly identified by a test. Sensitivity may be expressed as positive predictive value (PPV). Specificity may be expressed as negative predictive value (NPV).
PPV=number of samples from patients with disease tested positive/(number of samples from patients with disease tested positive+number of samples from patients without disease tested positive)
NPV=number of samples from patients without disease tested negative/(number of samples from patients with disease tested negative+number of samples from patients without disease tested negative)

Limit of detection (LOD) expresses analytical sensitivity or the ability of an assay to detect a target analyte (such as a Rickettsiaceae nucleic acid sequence), which is usually expressed as the minimum detectable concentration of the analyte. LOD can be expressed as a concentration of analyte, expressed in appropriate units describing a minimum concentration that can be detected by an assay or a detection method. “Analytical specificity” refers to the ability of an assay to measure a particular organism or substance, rather than others, in a sample.

The term “sequence” can be used to refer to the order of nucleotides in a nucleic acid, which can also be described as “primary structure,” or to a nucleic acid molecule, such as an oligonucleotide, with a particular base order.

“Sequence identity” or “sequence similarity” in the context of two or more nucleic acids sequences, refer to two or more sequences or subsequences that are the same or have a specified percentage nucleotides that are the same (for example, 60%, 65%, 70%, 75%, 80%, 85%, 90%, 91%, 92%, 93%, 94%, 95%, 96%, 97%, 98%, 99%, or higher identity) over a specified region, when compared and aligned for maximum correspondence over a comparison window or designated region. Various tools for measuring sequence similarity are available, such as a BLAST or BLAST 2.0 sequence comparison algorithms with default parameters available from NCBI or other sources. See also Altschul et al., Nuc. Acids Res. 25:3389-3402 (1977) and Altschul et al., J. Mol. Biol. 215:403-410 (1990). For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

“Target nucleic acid” is a nucleic acid molecule or sequence intended for one or more of amplification, detection, quantitation, quantitative, semi-quantitative or qualitative detection. The nucleic acid molecule need not be in an isolated form purified state. Various other nucleic acid molecules can also be present with the target nucleic acid molecule. For example, the target nucleic acid molecule can be a specific nucleic acid sequence. It can include RNA (such as Rickettsiaceae RNA) or DNA (such as genomic DNA or DNA produced by reverse transcription of RNA). In the context of the embodiments of the present invention, a target nucleic molecule can be a nucleic acid sequence corresponding to a region of Rickettsiaceae 23S rRNA, such as Rickettsia or Orientia 23S rRNA.

The embodiments of the present invention use PCR methods to detect target nucleic acids of Rickettsiaceae bacteria, such as Rickettsia and/or Orientia. “Real-time PCR” or rPCR is a method for detecting and measuring products generated during each cycle of a PCR, which are proportionate to the amount of template nucleic acid prior to the start of PCR. The information obtained, such as an amplification curve, can be used to determine the presence of a target nucleic acid (such as Rickettsiaceae nucleic acid, including Rickettsia and/or Orientia nucleic acid) and/or quantitate the initial amounts of a target nucleic acid sequence. The term “real-time PCR” is used to denote a subset of PCR techniques that allow for detection of PCR product throughout the PCR reaction, or in real-time. In some examples, rPCR is real time reverse transcriptase (RT) PCR (rRT-PCR). Reverse transcriptase PCR is used when the starting material is RNA. RNA is first transcribed into complementary DNA (cDNA) by reverse transcriptase. In rRT-PCR, the cDNA is then used as the template for the qPCR reaction. rRT-PCR can be performed in a one-step method, which combines reverse transcription and PCR in a single tube and buffer, using a reverse transcriptase along with a DNA polymerase. In one-step rRT-PCR, both RNA and DNA targets are amplified using sequence-specific targets. The term “quantitative PCR” encompasses all PCR-based techniques that allow for quantitative or semi-quantitative determination of the initially present target nucleic acid sequences.

The principles of real-time PCR (rPCR) are generally described, for example, in Held et al. “Real Time Quantitative PCR” Genome Research 6:986-994 (1996). Generally, rPCR measures a signal at each amplification cycle. Some rPCR techniques rely on fluorophores that emit a signal at the completion of every multiplication cycle. Examples of such fluorophores are fluorescence dyes that emit fluorescence at a defined wavelength upon binding to double-stranded DNA, such as SYBR green. An increase in double-stranded DNA during each amplification cycle thus leads to an increase in fluorescence intensity due to accumulation of PCR product. Another example of fluorophores used for detection in rPCR are sequence-specific fluorescent reporter probes, described elsewhere in this document. The examples of such probes are TaqMan® probes. The use of sequence-specific reporter probe provides for detection of a target sequence with high specificity, and enables quantification even in the presence of non-specific DNA amplification. Fluorescent probes can also be used in multiplex assays—for detection of several genes in the same reaction—based on specific probes with different-colored labels. For example, a multiplex assay can use several sequence-specific probes, labeled with a variety of fluorophores, including, but not limited to, FAM, JA270, CY5.5, and HEX, in the same PCR reaction mixture.

rPCR relies on detection of a measurable parameter, such as fluorescence, during the course of the PCR reaction. The amount of the measurable parameter is proportional to the amount of the PCR product, which allows one to observe the increase of the PCR product “in real time.” Some rPCR methods allow for quantification of the input DNA template based on the observable progress of the PCR reaction. The analysis and processing of the data is discussed below. A “growth curve” or “amplification curve” in the context of a nucleic acid amplification assay is a graph of a function, where an independent variable is the number of amplification cycles and a dependent variable is an amplification-dependent measurable parameter measured at each cycle of amplification, such as fluorescence emitted by a fluorophore. As discussed above, the amount of amplified target nucleic acid (such as Rickettsiaceae target nucleic acid, including Rickettsia and/or Orientia target nucleic acid) can be detected using a fluorophore-labeled probe. Typically, the amplification-dependent measurable parameter is the amount of fluorescence emitted by the probe upon hybridization, or upon the hydrolysis of the probe by the nuclease activity of the nucleic acid polymerase.

The increase in fluorescence emission is measured in real time and is directly related to the increase in target nucleic acid amplification (such as Rickettsiaceae target nucleic acid amplification). In some examples, the change in fluorescence (dRₙ) is calculated using the equation dRₙ=Rₙ₊−Rₙ₋, with Rₙ₊ being the fluorescence emission of the product at each time point and Rₙ₋ being the fluorescence emission of the baseline. The dRₙ values are plotted against cycle number, resulting in amplification plots. In a typical polymerase chain reaction, a growth curve contains a segment of exponential growth followed by a plateau, resulting in a sigmoidal-shaped amplification plot when using a linear scale. A growth curve is characterized by a “cross point” value or “Cₚ” value, which can be also termed “threshold value” or “cycle threshold” (Cₜ), which is a number of cycles where a predetermined magnitude of the measurable parameter is achieved. For example, when a fluorophore-labeled probe is employed, the threshold value (Cₜ) is the PCR cycle number at which the fluorescence emission (dRₙ) exceeds a chosen threshold, which is typically 10 times the standard deviation of the baseline (this threshold level can, however, be changed if desired). A lower Cₜ value represents more rapid completion of amplification, while the higher Cₜ value represents slower completion of amplification. Where efficiency of amplification is similar, the lower Cₜ value is reflective of a higher starting amount of the target nucleic acid, while the higher Cₜ value is reflective of a lower starting amount of the target nucleic acid. Where a control nucleic acid of known concentration is used to generate a “standard curve,” or a set of “control” Cₜ values at various known concentrations of a control nucleic acid, it becomes possible to determine the absolute amount of the target nucleic acid in the sample by comparing Cₜ values of the target and control nucleic acids.

Assays

Rickettsia

Embodiments of the present invention include real-time RT-PCR (rRT-PCR) assays useful for detection of a range of Rickettsia species. Thus, some of the assays according to the embodiments of the present invention can be referred to as “Rickettsia assays” or “pan-Rickettsia assays.” The primers of the pan-Rickettsia assays are useful for amplification of a region of Rickettsia 23 S ribosomal RNA nucleic acid sequence, which can be referred to as “23S rRNA target region.” The probes of the pan-Rickettsia assays are used for specific detection of the amplification products produced in the course of amplifying the 23S rRNA target region or a region of 23S rRNA comprising the sequence to which the probe sequence can hybridize. The assays, according to some embodiments of the present invention, amplify DNA from both genomic DNA and DNA generated from a reverse transcriptase reaction using rRNA target nucleic acid sequences and therefore can be conducted on total nucleic acid (TNA), DNA or RNA. For example, when contacted with the sample in the context of the rRT-PCR assay, the primers amplify a 23 S rRNA target region from genomic DNA and/or transcribed DNA from the rRNA, and the probes target the sequence within the amplified target region, generating a signal that can be interpreted to determine the presence or absence of target Rickettsia sequences in the sample. In one embodiment, the assay is conducted on TNA extracted from a biological sample. In another embodiment, the assay includes a step of extracting TNA from a biological sample. An example of a pan-Rickettsia assay according to an embodiment of the present invention employs a forward primer, a reverse primer and a probe labeled with a fluorescent label and a dark quencher for specific detection of a range of Rickettsia pathogenic to humans. One or both of the forward and the reverse primers can be the primers according to the embodiments of the present invention, but other suitable forward and reverse primers can be used. The probe can be a probe according to the embodiments of the present invention, but a different suitable probe can be used. One embodiment of a pan-Rickettsia assay is an RCKr assay described further in this document.

Rickettsia assays according to the embodiments of the present invention are designed to detect target region of Rickettsia 23S rRNA using one or more DNA primers and probes based on the sequences listed in Table 2. To perform Rickettsia assays according to the embodiments of the present invention, the primers and the probes can be combined in various ways discussed further in the document (see, for example, section “Kits” and Table 4). It is to be understood, of course, that the uses of the primer and the probe sequences described in this document, are not limited to the detection of Rickettsia in rRT-PCR assays, described in this document or elsewhere. The sequences shown in Table 2 can be used, separately and in various combinations amongst themselves and with other reagents, for example, to isolate, amplify or detect the nucleic acids containing one or more the relevant sequences, or their variants, to engineer various recombinant constructs and organisms and for other suitable uses. Some of these uses are described further in the sections “Probes” and “Primers.”

The assays according to the embodiments of the present invention can serve as effective tools for rapid and specific detection of Rickettsia in clinical and laboratory samples with high sensitivity, specificity and superior LOD. The assays of the present invention can have various application and uses. Tissue biopsies typically contain a higher concentration of Rickettsia, but the procedure is invasive and a specimen often difficult to obtain. Furthermore, a rash or eschar may not be present or visible at the time of sample collection. Blood and serum specimens are easier to collect, however the level of organism is generally low in these specimens, except in advance disease. The assay is able to increase accuracy of diagnosis by increasing sensitivity of rPCR detection. Using total nucleic acid (DNA and RNA) isolated from a sample to conduct the assay, as opposed to only DNA or only RNA, increases assay's target detection capacity, such as improving LOD. It was calculated that the assay is able to detect nucleic acids originating from a single Rickettsia organism in a 200 μL extract volume or 5 Rickettsia per 1 mL. Furthermore, the assays according to the embodiments of the present invention are accurate, quick and technologically accessible, meaning that they can be performed on-site or at the point of care using the compositions, kits and/or systems according to the embodiments of the present invention, and do not necessarily require sending the samples for testing to a remote location.

TABLE 2
Sequences used in the primer and probe
design or Rickettsia assays

| | | Location in |
|---|---|---|
| | | Rickettsia |
| | | 23S rRNA |
| | | sequence |
| Primer/Probe | Sequence (5′ > 3′) | (start)* |
| Rickettsia | GGT CCC ACA GAC TTA CCA AAC TCA | 874 |
| Forward | SEQ ID NO: 1 | |
| Primer 1 | | |
| Rickettsia | TCG ACT ATG GAC CTT AGC ACC CAT | 964 |
| Reverse | SEQ ID NO: 2 | |
| Primer | | |
| Rickettsia | CCG AAT GTC GAT GAG TAC AGC ATA GCA GAC | 906 |
| Probe 1 | SEQ ID NO: 3 | |
| Rickettsia | GGA TAT AGC TGG TTC TCC GCG AAA | 789 |
| Forward | SEQ ID NO: 4 | |
| Primer 2 | | |
| Rickettsia | TCG AAG GTA GAG CAC TGA ATG AGC | 845 |
| Forward | SEQ ID NO: 5 | |
| Primer 3 | | |
| Rickettsia | ACC ATC GAA GGT AGA GCA CTG AAT GAG C | 841 |
| Probe 2 | SEQ ID NO: 6 | |
| Rickettsia | GGG TCC CAC AGA CTT ACC AAA CTC AAT C | 873 |
| Probe 3 | SEQ ID NO:7 | |

*In reference to Rickettsia rickettsii strain Sheila Smith 23S ribosomal RNA gene, complete sequence, NCBI Reference Sequence: NR_103136.1

The assays according to the embodiments of the present invention can be conducted as singleplex assays or incorporated into multiplex assays. Singleplex assays are used to detect one target nucleic acid sequence, while multiplex assays allow one to detect several target nucleic acid sequences in the same assay. In a multiplex assay, the probes and/or the primers are uniquely labelled to distinguish their signals. Multiplex assays minimize the amount of starting material required, which can be of critical value when samples are limited. Multiplexing can also save time by increasing throughput and decreasing sample handling. It can also save on the cost of reagents and other consumables. For example, the assays according to the embodiments of the present invention can be multiplexed with rRT-PCR assays for other infectious agents that cause human disease, such as but not limited to, Orientia tsutsugamushi (scrub typhus agent), Coxiella burnetii (Q fever agent), Ehrlichia (ehrlichiosis agent) and Anaplasma (anaplasmosis agent). The above and other organisms may cause symptoms that are generic and similar to Rickettsia. Therefore, multiplexing assays that several such targets in a single assay is diagnostically advantageous by allowing for early differential diagnosis of a disease in a patient, as well as for accurate disease surveillance.

Orientia

Embodiments of the present invention also include real-time RT-PCR (rRT-PCR) assays useful for detection of Orientia, including Orientia tsutsugamushi. Thus, some of the assays according to the embodiments of the present invention can be referred to as “Orientia assays” or “scrub typhus assays.” The primers of Orientia assays are useful for amplification of a region of Orientia 23 S ribosomal RNA nucleic acid sequence, which can be referred to as “23 S rRNA target region.” The probes of the Orientia assays are used for specific detection of the amplification products produced in the course of amplifying the 23S rRNA target region or a region of 23S rRNA comprising the sequence to which the probe sequence can hybridize. The assays, according to some embodiments of the present invention, amplify DNA from both genomic DNA and DNA generated from a reverse transcriptase reaction using rRNA target nucleic acid sequences and therefore can be conducted on total nucleic acid (TNA), DNA or RNA. For example, when contacted with the sample in the context of the rRT-PCR assay, the primers amplify a 23S rRNA target region from genomic DNA and/or transcribed DNA from the rRNA, and the probes target the sequence within the amplified target region, generating a signal that can be interpreted to determine the presence or absence of target Orientia sequences in the sample. In one embodiment, the assay is conducted on TNA extracted from a biological sample. In another embodiment, the assay includes a step of extracting TNA from a biological sample. An example of Orientia assay according to an embodiment of the present invention employs a forward primer, a reverse primer and a probe labeled with a fluorescent label and a dark quencher for specific detection of Orientia in a sample. One or both of the forward and the reverse primers can be the primers according to the embodiments of the present invention, but other suitable forward and reverse primers can be used. The probe can be a probe according to the embodiments of the present invention, but a different suitable probe can be used. One embodiment of Orientia assay is an OTSr assay described further in this document

Orientia assays according to the embodiments of the present invention sensitively and specifically detect Orientia present in a sample. Analytical sensitivity of the Orientia assays according to the embodiments of the present invention allows them to detect low levels of Orientia. LOD of the Orientia assays according to the embodiments of the present invention allows them to detect low amounts of Orientia 23 S RNA nucleic acids (DNA, RNA or both DNA and RNA). LOD of Orientia assays according to the embodiments of the present invention can be at least 5, 6, 7, 8, 9, 10, 11, 12, 13, 14, 15, 16, 17 or 18 copies of target nucleic acid sequence per mL. LOD of some of the improved Orientia assays described in this document can be 10-100 times lower than the LOD of previously known Orientia assays.

Orientia assays according to the embodiments of the present invention are designed to detect target region of Orientia 23 S rRNA using one or more DNA primers and probes based on the sequences listed in Table 3. To perform Orientia assays according to the embodiments of the present invention, the primers and the probes can be combined in various ways discussed further in the document (see, for example, section “Kits” and Table 5). It is to be understood, of course, that the uses of the primer and the probe sequences described in this document, are not limited to the detection of Orientia in rRT-PCR assays, described in this document or elsewhere. The sequences shown in Table 3 can be used, separately and in various combinations amongst themselves and with other reagents, for example, to isolate, amplify or detect the nucleic acids containing one or more the relevant sequences, or their variants, to engineer various recombinant constructs and organisms and for other suitable uses. Some of these uses are described further in the sections “Probes” and “Primers.”

TABLE 3
Sequences used in the primer and probe design
of Orientia assays

| | | Location in Orientia |
|---|---|---|
| | | 23S rRNA sequence |
| Primer/Probe | Sequence (5′ > 3′) | (start)* |
| Orientia | CAA ACT CCG AAT GTC AAT AAA GT | 956 |
| Forward Primer 1 | SEQ ID NO: 8 | |
| Orientia | ATG ACG ATC TGG GCT GTT TC | 1,045 |
| Reverse Primer 1 | SEQ ID NO: 9 | |
| Orientia | TGT GGG CGC TAA GGT TCA TA | 997 |
| Probe 1 | SEQ ID NO: 10 | |
| Orientia | GGG GTC ATA CAG GCT TAC C | 928 |
| Forward Primer 2 | SEQ ID NO: 11 | |
| Orientia | TAT GAA CCT TAG CGC CCA CA | 1,016 |
| Reverse Primer 2 | SEQ ID NO: 12 | |
| Orientia | ACT TAA TCA AAC TCC GAA TGT CA | 949 |
| Probe 2 | SEQ ID NO: 13 | |
| Orientia | ACC AAA CTT AAT CAA ACT CCG A | 944 |
| Forward Primer 3 | SEQ ID NO: 14 | |
| Orientia | GGC TGT TTC CCT CTC GAC TA | 1,034 |
| Reverse Primer 3 | SEQ ID NO: 15 | |
| Orientia | GAC AGA CTG TGG GCG CTA A | 990 |
| Probe 3 | SEQ ID NO: 16 | |
| Orientia | TCA ATA AAG TAT AGC ATA ACA | 969 |
| Probe 4 | GAC AGA | |
| | SEQ ID NO: 17 | |
| Orientia | GAC AGA CTG TGG GCG CTA | 990 |
| Probe 5 | SEQ ID NO: 18 | |
| Orientia | CCA AAC TTA ATC AAA CTC CGA | 945 |
| Probe 6 | ATG TC | |
| | SEQ ID NO: 19 | |
| Orientia | AAC AGA CAG ACT GTG GGC GC | 986 |
| Probe 7 | SEQ ID NO: 20 | |

*In reference to Orientia tsutsugamushi strain Boryong 23S ribosomal RNA gene, complete sequence, NCBI Reference Sequence: NR_076246.1

The assays according to the embodiments of the present invention can serve as effective tools for rapid and specific detection of Orientia in clinical and laboratory samples with high sensitivity, specificity and superior LOD. The assays of the present invention can have various application and uses. Tissue biopsies typically contain a higher concentration of Orientia, but the procedure is invasive and a specimen often difficult to obtain. Furthermore, a rash or eschar may not be present or visible at the time of sample collection. Blood and serum specimens are easier to collect, however the level of organism is generally low in these specimens, except in advance disease. The assay is able to increase accuracy of diagnosis by increasing sensitivity of real-time PCR detection. Using total nucleic acid (DNA and RNA) isolated from a sample to conduct the assay, as opposed to only DNA or only RNA, increases assay's target detection capacity, such as improving LOD. It was calculated that the assay is able to detect nucleic acids originating from a single Orientia organism in a 200 μL extract volume or 5 Orientia per 1 mL. Furthermore, the assays according to the embodiments of the present invention are accurate, quick and technologically accessible, meaning that they can be performed on-site or at the point of care using the compositions, kits and/or systems according to the embodiments of the present invention, and do not necessarily require sending the samples for testing to a remote location.

The assays according to the embodiments of the present invention can be conducted as singleplex assays or incorporated into multiplex assays. Singleplex assays are used to detect one target nucleic acid sequence, while multiplex assays allow one to detect several target nucleic acid sequences in the same assay. In a multiplex assay, the probes and/or the primers are uniquely labelled to distinguish their signals. Multiplex assays minimize the amount of starting material required, which can be of critical value when samples are limited. Multiplexing can also save time by increasing throughput and decreasing sample handling. It can also save on the cost of reagents and other consumables. For example, the assays according to the embodiments of the present invention can be multiplexed with rRT-PCR assays for other infectious agents that cause human disease, such as but not limited to, Rickettsia, Coxiella burnetii (Q fever agent), Ehrlichia (ehrlichiosis agent) and Anaplasma (anaplasmosis agent). The above and other organisms may cause symptoms that are generic and similar to Orientia. Therefore, multiplexing assays that several such targets in a single assay is diagnostically advantageous by allowing for early differential diagnosis of a disease in a patient, as well as for accurate disease surveillance.

Probes

Some embodiments of the present invention are oligonucleotide probes. Some of the probes of the present invention are designed to hybridize to a region of Rickettsia 23S RNA nucleic acid sequence and are based on SEQ ID NOs 3, 6 or 7. These probes can be referred to as “Rickettsia probes,” “RCKr probes” and by other related terms. It is understood that the Rickettsia probes sequences are not limited to SEQ ID NOs 3, 6 or 7, but can include their variations, which can be defined based on sequence similarity. Some embodiments of the oligonucleotide probes can be employed to detect Rickettsia in rRT-PCR assays, including the assays according to the embodiments of the present invention, such as RCKr assay described in this document. However, the oligonucleotide probes described in this document are not limited to detection of Rickettsia in rRT-PCR assays and can have other uses, such as array detection, hybridization-based assays, for example, Northern or Southern blotting, in situ labelling, such us fluorescence in situ hybridization (FISH) and other uses.

The embodiments of the present invention include DNA probes suitable for detection of a region of Rickettsia 23S rRNA nucleic acid sequence that contains SEQ ID NO:3 or its variant. Some embodiments of Rickettsia probes contain (comprise) an oligonucleotide at least 85% identical to SEQ ID NO:3 (for example, an oligonucleotide at least 90% identical to SEQ ID NO:3, an oligonucleotide at least 95% identical to SEQ ID NO:3, or an oligonucleotide of SEQ ID NO:3). Some other embodiments consist of an oligonucleotide at least 85% identical to SEQ ID NO:3 (for example, an oligonucleotide at least 90% identical to SEQ ID NO:3, an oligonucleotide at least 95% identical to SEQ ID NO:3 or an oligonucleotide of SEQ ID NO:3) and reporting moieties discussed elsewhere in this document.

The embodiments of the present invention include DNA probes suitable for detection of a region of Rickettsia 23S rRNA nucleic acid sequence that contains SEQ ID NO:6 or its variant. Some embodiments of Rickettsia probes contain (comprise) an oligonucleotide at least 85% identical to SEQ ID NO:6 (for example, an oligonucleotide at least 90% identical to SEQ ID NO:6, an oligonucleotide at least 95% identical to SEQ ID NO:6, or an oligonucleotide of SEQ ID NO:6). Some other embodiments consist of an oligonucleotide at least 85% identical to SEQ ID NO:6 (for example, an oligonucleotide at least 90% identical to SEQ ID NO:6, an oligonucleotide at least 95% identical to SEQ ID NO:6 or an oligonucleotide of SEQ ID NO:6) and reporting moieties discussed elsewhere in this document.

The embodiments of the present invention include DNA probes suitable for detection of a region of Rickettsia 23S rRNA nucleic acid sequence that contains SEQ ID NO:7 or its variant. Some embodiments of Rickettsia probes contain (comprise) an oligonucleotide at least 85% identical to SEQ ID NO:7 (for example, an oligonucleotide at least 90% identical to SEQ ID NO:7, an oligonucleotide at least 95% identical to SEQ ID NO:7, or an oligonucleotide of SEQ ID NO:7). Some other embodiments consist of an oligonucleotide at least 85% identical to SEQ ID NO:7 (for example, an oligonucleotide at least 90% identical to SEQ ID NO:7, an oligonucleotide at least 95% identical to SEQ ID NO:7 or an oligonucleotide of SEQ ID NO:7) and reporting moieties discussed elsewhere in this document.

Some of the probes of the present invention are designed to hybridize to a region of Orientia 23S RNA nucleic acid sequence and are based on SEQ ID NOs 10, 13, 16, 17, 18, 19 and 20. These probes can be referred to as “Orientia probes” and by other related terms. It is understood that Orientia probes sequences are not limited to SEQ ID NOs 10, 13, 16, 17, 18, 19 and 20, but can include their variations, which can be defined based on sequence similarity. Some embodiments of the oligonucleotide probes can be employed to detect Orientia in rRT-PCR assays, including the assays according to the embodiments of the present invention, such as OTSr assay described in this document. However, the oligonucleotide probes described in this document are not limited to detection of Orientia in rRT-PCR assays and can have other uses, such as array detection, hybridization-based assays, for example, Northern or Southern blotting, in situ labelling, such us fluorescence in situ hybridization (FISH) and other uses.

The embodiments of the present invention include DNA probes suitable for detection of a region of Orientia 23S rRNA nucleic acid sequence that contains SEQ ID NO:10 or its variant. Some embodiments of Orientia probes contain (comprise) an oligonucleotide at least 85% identical to SEQ ID NO:10 (for example, an oligonucleotide at least 90% identical to SEQ ID NO:10, an oligonucleotide at least 95% identical to SEQ ID NO:10, or an oligonucleotide of SEQ ID NO:10). Some other embodiments consist of an oligonucleotide at least 85% identical to SEQ ID NO:10 (for example, an oligonucleotide at least 90% identical to SEQ ID NO:10, an oligonucleotide at least 95% identical to SEQ ID NO:10 or an oligonucleotide of SEQ ID NO:10) and reporting moieties discussed elsewhere in this document.

The embodiments of the present invention include DNA probes suitable for detection of a region of Orientia 23S rRNA nucleic acid sequence that contains SEQ ID NO:13 or its variant. Some embodiments of Orientia probes contain (comprise) an oligonucleotide at least 85% identical to SEQ ID NO:13 (for example, an oligonucleotide at least 90% identical to SEQ ID NO:13, an oligonucleotide at least 95% identical to SEQ ID NO:13, or an oligonucleotide of SEQ ID NO:13). Some other embodiments consist of an oligonucleotide at least 85% identical to SEQ ID NO:13 (for example, an oligonucleotide at least 90% identical to SEQ ID NO:13, an oligonucleotide at least 95% identical to SEQ ID NO:13 or an oligonucleotide of SEQ ID NO:13) and reporting moieties discussed elsewhere in this document.

The embodiments of the present invention include DNA probes suitable for detection of a region of Orientia 23S rRNA nucleic acid sequence that contains SEQ ID NO:16 or its variant. Some embodiments of Orientia probes contain (comprise) an oligonucleotide at least 85% identical to SEQ ID NO:16 (for example, an oligonucleotide at least 90% identical to SEQ ID NO:16, an oligonucleotide at least 95% identical to SEQ ID NO:16, or an oligonucleotide of SEQ ID NO:16). Some other embodiments consist of an oligonucleotide at least 85% identical to SEQ ID NO:16 (for example, an oligonucleotide at least 90% identical to SEQ ID NO:16, an oligonucleotide at least 95% identical to SEQ ID NO:16 or an oligonucleotide of SEQ ID NO:16) and reporting moieties discussed elsewhere in this document.

The embodiments of the present invention include DNA probes suitable for detection of a region of Orientia 23S rRNA nucleic acid sequence that contains SEQ ID NO:17 or its variant. Some embodiments of Orientia probes contain (comprise) an oligonucleotide at least 85% identical to SEQ ID NO:17 (for example, an oligonucleotide at least 90% identical to SEQ ID NO:17, an oligonucleotide at least 95% identical to SEQ ID NO:17, or an oligonucleotide of SEQ ID NO:17). Some other embodiments consist of an oligonucleotide at least 85% identical to SEQ ID NO:17 (for example, an oligonucleotide at least 90% identical to SEQ ID NO:17, an oligonucleotide at least 95% identical to SEQ ID NO:17 or an oligonucleotide of SEQ ID NO:17) and reporting moieties discussed elsewhere in this document.

The embodiments of the present invention include DNA probes suitable for detection of a region of Orientia 23S rRNA nucleic acid sequence that contains SEQ ID NO:18 or its variant. Some embodiments of Orientia probes contain (comprise) an oligonucleotide at least 85% identical to SEQ ID NO:18 (for example, an oligonucleotide at least 90% identical to SEQ ID NO:18, an oligonucleotide at least 95% identical to SEQ ID NO:18, or an oligonucleotide of SEQ ID NO:18). Some other embodiments consist of an oligonucleotide at least 85% identical to SEQ ID NO:18 (for example, an oligonucleotide at least 90% identical to SEQ ID NO:18, an oligonucleotide at least 95% identical to SEQ ID NO:18 or an oligonucleotide of SEQ ID NO:18) and reporting moieties discussed elsewhere in this document.

The embodiments of the present invention include DNA probes suitable for detection of a region of Orientia 23S rRNA nucleic acid sequence that contains SEQ ID NO:19 or its variant. Some embodiments of Orientia probes contain (comprise) an oligonucleotide at least 85% identical to SEQ ID NO:19 (for example, an oligonucleotide at least 90% identical to SEQ ID NO:19, an oligonucleotide at least 95% identical to SEQ ID NO:19, or an oligonucleotide of SEQ ID NO:19). Some other embodiments consist of an oligonucleotide at least 85% identical to SEQ ID NO:19 (for example, an oligonucleotide at least 90% identical to SEQ ID NO:19, an oligonucleotide at least 95% identical to SEQ ID NO:19 or an oligonucleotide of SEQ ID NO:19) and reporting moieties discussed elsewhere in this document.

The embodiments of the present invention include DNA probes suitable for detection of a region of Orientia 23S rRNA nucleic acid sequence that contains SEQ ID NO:20 or its variant. Some embodiments of Orientia probes contain (comprise) an oligonucleotide at least 85% identical to SEQ ID NO:20 (for example, an oligonucleotide at least 90% identical to SEQ ID NO:20, an oligonucleotide at least 95% identical to SEQ ID NO:20, or an oligonucleotide of SEQ ID NO:20). Some other embodiments consist of an oligonucleotide at least 85% identical to SEQ ID NO:20 (for example, an oligonucleotide at least 90% identical to SEQ ID NO:20, an oligonucleotide at least 95% identical to SEQ ID NO:20 or an oligonucleotide of SEQ ID NO:20) and reporting moieties discussed elsewhere in this document.

The length of a probe according to the embodiments of the present invention depends on the primers selected for a particular rRT-PCR assay and other factors, such as probe chemistry and sequence composition. An exemplary probe can be 20-40 bp long. For example a probe can be 25-35 bp long, about 30 bp long (meaning 15±3, 15±2, 1±1 bp long) 26, 27, 28 or 29 bp long). A probe is designed with about 6-8° C. higher Tₘ than Tₘs of the primers. A probe typically contains reporting moieties and may contain other moieties, such as linkers, stabilizers, modified bases, etc., the selection of which depends on a probe chemistry. In one example, the probe can be a TaqMan® probe labeled with a fluorophore moiety, such as FAM, and a quencher moiety, but other types of probe chemistries can be employed. In an exemplary TaqMan® probe, the fluorophore moiety is coupled to 5′ terminus of the probe. One example of a suitable fluorophore is a fluorescein moiety, such as FAM. One example of a suitable quencher is a dark quencher, for example BHQ quencher, such as BHQ1. The quencher can be coupled to 3′ terminus of the probe or to an internal base. The probe can also contain a duplex stabilizer. Some embodiments of the probes incorporate MGB moieties and/or modified bases.

Rickettsia probes according to the embodiments of the present invention include the probes suitable for RCKr assay, which can be referred to as “RCKr probes.” One exemplary embodiment of a RCKr probe is a probe consisting of SEQ ID NO:3 oligonucleotide, FAM fluorophore coupled to 5′ terminus and BHQ1 quencher coupled to 5′ end of the oligonucleotide, as shown in Table 6. One more exemplary embodiment of a RCKr probe is a probe consisting of SEQ ID NO:6 oligonucleotide, FAM fluorophore coupled to 5′ terminus and BHQ1 quencher coupled to 5′ end of the oligonucleotide. One more exemplary embodiment of a RCKr probe is a probe consisting of SEQ ID NO:7 oligonucleotide, FAM fluorophore coupled to 5′ terminus and BHQ1 quencher coupled to 5′ end of the oligonucleotide. Orientia probes according to the embodiments of the present invention include the probes suitable for OTSr assay, which can be referred to as “OTSr probes.” One exemplary embodiment of an OTSr probe is a probe consisting of SEQ ID NO:10 oligonucleotide, FAM fluorophore coupled to 5′ terminus and BHQ1 quencher coupled to 5′ end of the oligonucleotide, as shown in Table 12. One more exemplary embodiment of an OTSr probe is a probe consisting of SEQ ID NO:13 oligonucleotide, FAM fluorophore coupled to 5′ terminus and BHQ1 quencher coupled to 5′ end of the oligonucleotide. One more exemplary embodiment of an OTSr probe is a probe consisting of SEQ ID NO:16 oligonucleotide, FAM fluorophore coupled to 5′ terminus and BHQ1 quencher coupled to 5′ end of the oligonucleotide. One more exemplary embodiment of an OTSr probe is a probe consisting of SEQ ID NO:17 oligonucleotide, FAM fluorophore coupled to 5′ terminus and BHQ1 quencher coupled to 5′ end of the oligonucleotide. One more exemplary embodiment of an OTSr probe is a probe consisting of SEQ ID NO:18 oligonucleotide, FAM fluorophore coupled to 5′ terminus and BHQ1 quencher coupled to 5′ end of the oligonucleotide. One more exemplary embodiment of an OTSr probe is a probe consisting of SEQ ID NO:19 oligonucleotide, FAM fluorophore coupled to 5′ terminus and BHQ1 quencher coupled to 5′ end of the oligonucleotide. One more exemplary embodiment of an OTSr probe is a probe consisting of SEQ ID NO:20 oligonucleotide, FAM fluorophore coupled to 5′ terminus and BHQ1 quencher coupled to 5′ end of the oligonucleotide. Some of the above probe embodiments incorporate MGB moieties and/or modified bases. Other exemplary embodiments of probes are a ZEN® Double-Quenched Probe (manufactured by Integrated DNA Technologies, Coraville, Iowa) or a QSY® probe (ThermoFisher Scientific, Waltham, Mass.) comprising SEQ ID NO:3, 6, 7, 10, 13, 16, 17, 18, 19 or 20. It is to be understood that the choice of a fluorophore and quencher for a particular probe depends on the type of a probe and probe design.

Primers

Embodiments of the present invention include DNA oligonucleotides that can be employed for amplification of a region of Rickettsia 23S rRNA nucleic acid sequence. Some embodiments of the oligonucleotide primers can be employed in Rickettsia rRT-PCR assays, including the assays according to the embodiments of the present invention, such as RCKr assay described in this document. The uses of the primers described in this document are not limited to such assays; the primers can be used to amplify any nucleic acids containing regions having sequence similarity to the primer sequences shown in Table 2. The uses of the primers according to the embodiments of the present invention are also not limited to PCR amplification, such as rRT-PCR assays; the primers can be used in various other assays and methods, for example, in sequencing or array-based detection.

Some of the primers according to the embodiments of the present invention are based on SEQ ID NOs 1, 2, 4 or 5, which are shown in Table 2. Some embodiments of the primers contain (comprise) an oligonucleotide at least 85% identical to SEQ ID NOs 1, 2, 4 or 5 (for example, an oligonucleotide at least 90% identical to SEQ ID NOs 1, 2, 4 or 5, an oligonucleotide at least 95% identical to SEQ ID NOs 1, 2, 4 or 5, an oligonucleotide 99% identical to SEQ ID NOs 1, 2, 4 or 5, or an oligonucleotide of SEQ ID NOs 1, 2, 4 or 5). The primers are suitable for amplification of a region of Rickettsia 23S RNA nucleic acid sequence by a polymerase chain reaction. For example, the primers for PCR amplification of Rickettsia 23S RNA nucleic acid sequence can include a “forward” primer comprising an oligonucleotide at least 90% identical SEQ ID NO:1 and a “reverse” primer comprising an oligonucleotide at least 90% identical SEQ ID NO:2. For example, a forward primer can be an oligonucleotide comprising SEQ ID NO:1 or its variant, an oligonucleotide consisting of SEQ ID NO:1 or its variant, or oligonucleotide consisting of SEQ ID NO:1 or its variant and reporting moieties or labels. A reverse primer can be an oligonucleotide comprising SEQ ID NO:2 or its variant, an oligonucleotide consisting of SEQ ID NO:2 or its variant, or oligonucleotide consisting of SEQ ID NO:2 or its variant and reporting moieties or labels. It is understood that, for amplification of a region of Rickettsia 23S RNA nucleic acid sequence or for other uses, forward and reverse primers can be used together as a primer pair, but can also be used separately in combination with the other primers. For example, the forward primer based on SEQ ID NOs 1, 4 or 5 can be combined with the reverse primer based on SEQ ID NO:2 for amplification of a region Rickettsia 23S RNA nucleic acid sequence, but can also be combined with a suitable primer other than the reverse primer based on SEQ ID NO:2. Likewise, the reverse primer based on SEQ ID NO:2 can be combined with the forward primers based on SEQ ID NOs 1, 4 or 5 for amplification of a region Rickettsia 23S RNA nucleic acid sequence, but the reverse primer based on SEQ ID NO:2 can also be combined with a suitable primer other than the forward primers based on SEQ ID NOs 1, 4 or 5.

Embodiments of the present invention include DNA oligonucleotides that can be employed for amplification of a region of Orientia 23S rRNA nucleic acid sequence. Some embodiments of the oligonucleotide primers can be employed in Orientia rRT-PCR assays, including the assays according to the embodiments of the present invention, such as OTSr assay described in this document. The uses of the primers described in this document are not limited to such assays; the primers can be used to amplify any nucleic acids containing regions having sequence similarity to the primer sequences shown in Table 3. The uses of the primers according to the embodiments of the present invention are also not limited to PCR amplification, such as rRT-PCR assays; the primers can be used in various other assays and methods, for example, in sequencing or array-based detection.

Some of the primers according to the embodiments of the present invention are based on SEQ ID NOs 8, 9, 11, 12, 14 and 15, which are shown in Table 3. Some embodiments of the primers contain (comprise) an oligonucleotide at least 85% identical to SEQ ID NOs 8, 9, 11, 12, 14 and 15 (for example, an oligonucleotide at least 90% identical to SEQ ID NOs 8, 9, 11, 12, 14 and 15, an oligonucleotide at least 95% identical to SEQ ID NOs 8, 9, 11, 12, 14 and 15, an oligonucleotide 99% identical to SEQ ID NOs 8, 9, 11, 12, 14 and 15, or an oligonucleotide of SEQ ID NOs 8, 9, 11, 12, 14 and 15). The primers are suitable for amplification of a region of Orientia 23S RNA nucleic acid sequence by a polymerase chain reaction. For example, the primers for PCR amplification of a region of Orientia 23S RNA nucleic acid sequence can include a “forward” primer comprising an oligonucleotide at least 90% identical SEQ ID NOs 8, 11 or 14, and a “reverse” primer comprising an oligonucleotide at least 90% identical SEQ ID NOs 9, 12 or 15. For example, a forward primer can be an oligonucleotide comprising SEQ ID NO:8 or its variant, an oligonucleotide consisting of SEQ ID NO:8 or its variant, or oligonucleotide consisting of SEQ ID NO:8 or its variant and reporting moieties or labels. In another example, a forward primer can be an oligonucleotide comprising SEQ ID NO:11 or its variant, an oligonucleotide consisting of SEQ ID NO:11 or its variant, or oligonucleotide consisting of SEQ ID NO:11 or its variant and reporting moieties or labels. In one more example, a forward primer can be an oligonucleotide comprising SEQ ID NO:14 or its variant, an oligonucleotide consisting of SEQ ID NO:14 or its variant, or oligonucleotide consisting of SEQ ID NO:14 or its variant and reporting moieties or labels. A reverse primer can be an oligonucleotide comprising SEQ ID NO:9 or its variant, an oligonucleotide consisting of SEQ ID NO:9 or its variant, or oligonucleotide consisting of SEQ ID NO:9 or its variant and reporting moieties or labels. In another example, a reverse primer can be an oligonucleotide comprising SEQ ID NO:12 or its variant, an oligonucleotide consisting of SEQ ID NO:12 or its variant, or oligonucleotide consisting of SEQ ID NO:12 or its variant and reporting moieties or labels. In yet another example, a reverse primer can be an oligonucleotide comprising SEQ ID NO:15 or its variant, an oligonucleotide consisting of SEQ ID NO:15 or its variant, or oligonucleotide consisting of SEQ ID NO:15 or its variant and reporting moieties or labels. It is understood that, for amplification of a region of Orientia 23S RNA nucleic acid sequence or for other uses, forward and reverse primers can be used together as a primer pair, but can also be used separately in combination with the other primers. For example, a forward primer based on SEQ ID NO 8 can be combined with a reverse primer based on SEQ ID NOs 9 or 15 for amplification of a region Orientia 23S RNA nucleic acid sequence, but can also be combined with a suitable primer other than the above reverse primer. In another example, a forward primer based on SEQ ID NO 11 can be combined with a reverse primer based on SEQ ID NOs 9, 12 or 15 for amplification of a region Orientia 23S RNA nucleic acid sequence, but can also be combined with a suitable primer other than the above reverse primer. In one more example, a forward primer based on SEQ ID NO 14 can be combined with a reverse primer based on SEQ ID NOs 9 or 15 for amplification of a region Orientia 23S RNA nucleic acid sequence, but can also be combined with a suitable primer other than the above reverse primer. In another example, a reverse primer based on SEQ ID NO 9 can be combined with a forward primer based on SEQ ID NOs 8, 11 or 14 for amplification of a region Orientia 23S RNA nucleic acid sequence, but a reverse primer based on SEQ ID NO:9 can also be combined with a suitable primer other than the above forward primer. In yet another example, a reverse primer based on SEQ ID NO 12 can be combined with a forward primer based on SEQ ID NOs 11 or 14 for amplification of a region Orientia 23S RNA nucleic acid sequence, but the reverse primer based on SEQ ID NO 12 can also be combined with a suitable primer other than the above forward primer. In one more example, a reverse primer based on SEQ ID NO 15 can be combined with a forward primer based on SEQ ID NOs 8, 11 or 14 for amplification of a region Orientia 23S RNA nucleic acid sequence, but a reverse primer based on SEQ ID NO 15 can also be combined with a suitable primer other than the above forward primer.

It is to be understood that the primers according to the embodiments of the present invention can be unmodified and unlabeled DNA oligonucleotides. The primers according to the embodiments of the present invention can also contain reporting or labelling moieties, such as fluorescent moieties, quencher moieties or their combinations. The primers according to the embodiments of the present invention can also contain unnatural and modified nucleotides, linkers and other moieties. The length of the primers can vary. For example, the primers can be 15-35 bp long. A primer length is selected to be long enough for adequate specificity and short enough for primers to bind easily to the target nucleic acid at the annealing temperature. For example, the primers can be 18-30 bp long, for example, 20, 21, 22, 23, 24, 35, 26, 27, 28, 29, or 30 bp long. A primer is designed to have a Tₘ that is 6-8° C. lower than Tm of the probe, yet sufficiently high to ensure specific binding. An exemplary primer can have a Tm of about 55-65° C., for example, about 58, 59 or 60° C., but TₘS outside of this range are also possible, depending on the specific primer.

Kits

The embodiments of the present invention also include kits comprising one or more of the primers and/or the probes described above. In other words, the primers and/or the probes according to the embodiments of the present invention can be included or combined, in various ways, in kits. Such kits can be used for detection, including semi-quantitative and quantitative detection, of Rickettsia and/or Orientia in samples, such as the samples derived from human or animal subjects, laboratory samples, nucleic acid isolate samples, etc. It is to be understood that at least some of the kits described in this document are not limited to Rickettsia and/or Orientia detection and can be generally used to detect and/or amplify nucleic acids containing the sequences used in the design or the probes included in the kits. These sequences are shown in Tables 2 and 3.

Some embodiments of the present invention provide the kits for performing Rickettsia assays according to the embodiments of the present invention. Such kits comprise primers and probes that can be combined in various ways, such as those shown in Table 4. It is to be understood that other combinations, as well as other primers and probes not listed in Table 4 and/or described in this document, can be employed in the kits along with the primers and the probes described herein. It is also to be understood that such kits can include other ingredients and/or devices for performing Rickettsia assays according to the embodiments of the present invention, such as reagents for performing rRT-PCR assays.

TABLE 4
Examples of possible combinations of primers and
probes for kits and/or Rickettsia assays according
to the embodiments of the present invention

| | Forward primer | Reverse primer | |
|---|---|---|---|
| | based on: | based on: | Probe based on: |
| Combination 1 | SEQ ID NO: 1 | SEQ ID NO: 2 | SEQ ID NO: 3 |
| Combination 2 | SEQ ID NO: 4 | SEQ ID NO: 2 | SEQ ID NO: 3 |
| Combination 3 | SEQ ID NO: 5 | SEQ ID NO: 2 | SEQ ID NO: 3 |
| Combination 4 | SEQ ID NO: 4 | SEQ ID NO: 2 | SEQ ID NO: 6 |

Some other embodiments of the present invention provide the kits for amplifying Rickettsia 23S rRNA nucleic acid sequences (including RNA and genomic DNA sequences). Such kits comprise primers according to the embodiments of the present invention, which can be combined in ways discussed in the section “Primers.” It is to be understood that other combinations, as well as other primers not described in this document, can be employed in the kits along with the primers described herein. Furthermore, such kits can comprise additional ingredients and/or devices for performing nucleic acid amplifications, such as PCR reagents.

Some examples of the kit embodiments are described below. One or more Rickettsia probes according to the embodiments of the present invention (see, for example, section “Probes”) can be included in the kits useful for detecting Rickettsia by rRT-PCR assays. For example, one or more Rickettsia probes according to the embodiments of the present invention can be included in a kit along with other reagents for performing an rRT-PCR assay. Such a kit can be used for detecting Rickettsia in the sample. The other reagents included in the kits can include one or more PCR primers, such as one or more primers according to the embodiments of the present invention. For example, a kit can include a probe based on SEQ ID NO:3, and one or more of a forward primer based on SEQ ID NOs 1, 4 or 5 and a reverse primer based on SEQ ID NO:2. In another non-limiting example, a kit can include a probe based on SEQ ID NO:6, and one or more of a forward primer based on SEQ ID NO:4 and a reverse primer based on SEQ ID NO:2.

Some embodiments of the present invention provide the kits for performing Orientia assays according to the embodiments of the present invention. Such kits comprise primers and probes that can be combined in various ways, such as those shown in Table 5. It is to be understood that other primers and probes not listed in Table 5 and/or described in this document, can be employed in the kits along with the primers and the probes described herein. It is also to be understood that such kits can include other ingredients and/or devices for performing Orientia assays according to the embodiments of the present invention, such as reagents for performing rRT-PCR assays.

Some other embodiments of the present invention provide the kits for amplifying Orientia 23S rRNA nucleic acid sequences (including RNA and genomic DNA sequences). Such kits comprise primers according to the embodiments of the present invention, which can be combined in ways discussed in the section “Primers.” It is to be understood that other combinations, as well as other primers not described in this document, can be employed in the kits along with the primers described herein. Furthermore, such kits can comprise additional ingredients and/or devices for performing nucleic acid amplifications, such as PCR reagents.

Some examples of the kit embodiments are described below. One or more Orientia probes according to the embodiments of the present invention (see, for example, section “Probes”) can be included in the kits useful for detecting Orientia by rRT-PCR assays. For example, one or more Orientia probes according to the embodiments of the present invention can be included in a kit along with other reagents for performing an rRT-PCR assay. Such a kit can be used for detecting Orientia in the sample. The other reagents included in the kits can include one or more PCR primers, such as one or more primers according to the embodiments of the present invention. For example, a kit can include one or more probes based on SEQ ID NOs 10, 17, 18, 19 or 20 and one or both of a forward primer based on SEQ ID NO:8 and a reverse primer based on SEQ ID NO:9. In another non-limiting example, a kit can include a probe based on SEQ ID NO:13, and one or both of a forward primer based on SEQ ID NO:11 and a reverse primer based on SEQ ID NO:12. In one more non-limiting example, a kit can include a probe based on SEQ ID NO:16, and one or both of a forward primer based on SEQ ID NO:14 and a reverse primer based on SEQ ID NO:15. In yet one more non-limiting example, a kit can include a probe based on SEQ ID NO:16 and one or both of a forward primer based on SEQ ID NO:8 and a reverse primer based on SEQ ID NO:9 or SEQ ID NO:15. In one more non-limiting example, a kit can include a probe based on SEQ ID NO:16 and one or both of a forward primer based on SEQ ID NO:11 or SEQ ID NO:14 and a reverse primer based on SEQ ID NO:9. In one more non-limiting example, a kit can include a probe based on SEQ ID NO:16 and one or both of a forward primer based on SEQ ID NO:11 or SEQ ID NO:11 and a reverse primer based on SEQ ID NO:15. In one more non-limiting example, a kit can include a probe based on SEQ ID NO:18 and one or both of a forward primer based on SEQ ID NO:15 and a reverse primer based on SEQ ID NO:15. In one more non-limiting example, a kit can include a probe based on SEQ ID NO:18 and one or both of a forward primer based on SEQ ID NO:11 and a reverse primer based on SEQ ID NO:9 or SEQ ID NO:15. In one more non-limiting example, a kit can include a probe based on SEQ ID NO:18 and one or both of a forward primer based on SEQ ID NO:14 and a reverse primer based on SEQ ID NO:9 or SEQ ID NO:15. In yet another non-limiting example, a kit can include a probe based on SEQ ID NO:20 and one or both of a forward primer based on SEQ ID NO:8 or SEQ ID NO:11 and a reverse primer based on SEQ ID NO:15. In yet another non-limiting example, a kit can include a probe based on SEQ ID NO:20 and one or both of a forward primer based on SEQ ID NO:14 and a reverse primer based on SEQ ID NO:9 or SEQ ID NO:15. In one more non-limiting example, a kit can include a probe based on SEQ ID NO:20 and one or both of a forward primer based on SEQ ID NO:11 and a reverse primer based on SEQ ID NO:9. In one more non-limiting example, a kit can include a probe based on SEQ ID NO:10 and one or both of a forward primer based on SEQ ID NO:11 or SEQ ID NO:14 and a reverse primer based on SEQ ID NO:9. In one more non-limiting example, a kit can include a probe based on SEQ ID NO:13 and one or both of a forward primer based on SEQ ID NO:11 and a reverse primer based on SEQ ID NO:9 or SEQ ID NO:15. In one more non-limiting example, a kit can include a probe based on SEQ ID NO:17 and one or both of a forward primer based on SEQ ID NO:11 and a reverse primer based on SEQ ID NO:9, SEQ ID NO:12 or SEQ ID NO:15. In one more non-limiting example, a kit can include a probe based on SEQ ID NO:17 and one or both of a forward primer based on SEQ ID NO:14 and a reverse primer based on SEQ ID NO:9, SEQ ID NO:12 or SEQ ID NO:15.

TABLE 5
Examples of possible combinations of primers and probes for kits and/or
Orientia assays according to the embodiments of the present invention

| | Forward primer | Reverse primer | |
|---|---|---|---|
| | based on: | based on: | Probe based on: |
| Combination 1 | SEQ ID NO: 8 | SEQ ID NO: 9 | SEQ ID NO: 10 |
| Combination 2 | SEQ ID NO: 11 | SEQ ID NO: 12 | SEQ ID NO: 13 |
| Combination 3 | SEQ ID NO: 14 | SEQ ID NO: 15 | SEQ ID NO: 16 |
| Combination 4 | SEQ ID NO: 8 | SEQ ID NO: 9 | SEQ ID NO: 17 |
| Combination 5 | SEQ ID NO: 8 | SEQ ID NO: 9 | SEQ ID NO: 18 |
| Combination 6 | SEQ ID NO: 8 | SEQ ID NO: 9 | SEQ ID NO: 19 |
| Combination 7 | SEQ ID NO: 8 | SEQ ID NO: 9 | SEQ ID NO: 20 |
| Combination 8 | SEQ ID NO: 8 | SEQ ID NO: 9 | SEQ ID NO: 16 |
| Combination 9 | SEQ ID NO: 8 | SEQ ID NO: 15 | SEQ ID NO: 16 |
| Combination 10 | SEQ ID NO: 8 | SEQ ID NO: 15 | SEQ ID NO: 18 |
| Combination 11 | SEQ ID NO: 8 | SEQ ID NO: 15 | SEQ ID NO: 20 |
| Combination 12 | SEQ ID NO: 11 | SEQ ID NO: 9 | SEQ ID NO: 10 |
| Combination 13 | SEQ ID NO: 11 | SEQ ID NO: 9 | SEQ ID NO: 13 |
| Combination 14 | SEQ ID NO: 11 | SEQ ID NO: 9 | SEQ ID NO: 16 |
| Combination 15 | SEQ ID NO: 11 | SEQ ID NO: 9 | SEQ ID NO: 17 |
| Combination 16 | SEQ ID NO: 11 | SEQ ID NO: 9 | SEQ ID NO: 18 |
| Combination 17 | SEQ ID NO: 11 | SEQ ID NO: 9 | SEQ ID NO: 20 |
| Combination 18 | SEQ ID NO: 11 | SEQ ID NO: 12 | SEQ ID NO: 17 |
| Combination 19 | SEQ ID NO: 11 | SEQ ID NO: 15 | SEQ ID NO: 13 |
| Combination 20 | SEQ ID NO: 11 | SEQ ID NO: 15 | SEQ ID NO: 16 |
| Combination 21 | SEQ ID NO: 11 | SEQ ID NO: 15 | SEQ ID NO: 17 |
| Combination 22 | SEQ ID NO: 11 | SEQ ID NO: 15 | SEQ ID NO: 18 |
| Combination 23 | SEQ ID NO: 11 | SEQ ID NO: 15 | SEQ ID NO: 20 |
| Combination 24 | SEQ ID NO: 14 | SEQ ID NO: 9 | SEQ ID NO: 10 |
| Combination 25 | SEQ ID NO: 14 | SEQ ID NO: 9 | SEQ ID NO: 16 |
| Combination 26 | SEQ ID NO: 14 | SEQ ID NO: 9 | SEQ ID NO: 17 |
| Combination 27 | SEQ ID NO: 14 | SEQ ID NO: 9 | SEQ ID NO: 18 |
| Combination 28 | SEQ ID NO: 14 | SEQ ID NO: 9 | SEQ ID NO: 20 |
| Combination 29 | SEQ ID NO: 14 | SEQ ID NO: 12 | SEQ ID NO: 17 |
| Combination 30 | SEQ ID NO: 14 | SEQ ID NO: 15 | SEQ ID NO: 17 |
| Combination 31 | SEQ ID NO: 14 | SEQ ID NO: 15 | SEQ ID NO: 18 |
| Combination 32 | SEQ ID NO: 14 | SEQ ID NO: 15 | SEQ ID NO: 20 |

Some embodiments of the present invention provide the kits for performing both Rickettsia and Orientia assays according to the embodiments of the present invention. Such kits can be referred to as “combination kits” and can comprise one or more primers and probes for performing Rickettsia assays described in this document, as well as one or more primers and probes for performing Orientia assays described in this document. The primers and probes in such kits can be combined in various ways, including the combinations shown in Tables 4 and 5. Such kits can include other ingredients and/or devices for performing the assays according to the embodiments of the present invention, such as reagents for performing rRT-PCR assays. Some other embodiments of the present invention provide the kits for amplifying Rickettsia and Orientia, which can be collectively referred to as Rickettsiaceae, 23S rRNA nucleic acid sequences (including RNA and genomic DNA sequences). Such kits can comprise primers according to the embodiments of the present invention, which can be combined in ways discussed in the section “Primers.” It is to be understood that other combinations, as well as other primers not described in this document, can be employed in the kits along with the primers presently described. Furthermore, such kits can comprise additional ingredients and/or devices for performing nucleic acid amplifications, such as PCR reagents.

Some of the kits according to the embodiments of the present invention can include additional reagents for performing an rRT-PCR assays. The examples of additional reagents are enzymes for performing rRT-PCR assays are reverse transcriptase, DNA polymerase, such as Taq polymerase, PCR buffers, dNTPs and various additives, such as the additives that allow for efficient amplification of GC-rich templates. Some other examples of possible additional reagents are DNA-binding dyes, such as SYBR Green or EvaGreen, which can be employed in rRT-PCR assays that employ unlabeled primers and no probes. Some other examples of possible additional reagents are reagents used for nucleic acid isolation, such as the reagents used for total nucleic acid isolation, for example, cell and/or tissue lysis reagents, nucleic acid binding, nucleic acid elution buffers, proteinases, binding resins etc. The kits according to the embodiments of the present invention may also comprise devices, apparatuses and/or instruments used for sample preparation and/or storage, such as cell and/or tissue sample holding and/or preservation devices, nucleic acid extraction instruments, filters and/or chromatography columns.

Methods

Embodiments of the present invention also include methods of using the primers, probes and/or kits described above (“method embodiments”). The rPCR assays described throughout this document is an example of such methods. Some other method embodiments are methods of amplifying a region of Rickettsia and/or Orientia (which can be collectively referred to as Rickettsiaceae) 23S rRNA nucleic acid sequence by a PCR using one or more of the primers described in this document. Such methods can be referred to as “methods of amplifying Rickettsiaceae 23 S rRNA nucleic acid” “methods of amplifying a Rickettsiaceae 23S rRNA nucleic acid sequence,” “methods of amplifying a region of Rickettsiaceae 23S rRNA nucleic acid sequence” “amplification methods,” and by other related expressions. The method embodiments can include a step of contacting a sample, which may contain Rickettsiaceae 23S rRNA nucleic acid sequences, with one or more primers described in this document. After the contacting step, a nucleic acid amplification reaction, for example, PCR amplification (such as rRT-PCR, discussed in more detail elsewhere in this document or a different type of PCR), is performed under suitable conditions and using suitable reagents, and the amplification products can be detected by various detection procedures. The amplification methods can be used to determine if a nucleic acid sequence corresponding to Rickettsiaceae (Rickettsia and/or Orientia) 23S rRNA nucleic acid sequence is present in the sample, based on the detection of one or more products of the amplification.

Some other method embodiments are methods of amplifying a region of Rickettsia 23S rRNA nucleic acid sequence by a PCR using one or more of the primers described in this document. Such methods can be referred to as “methods of amplifying Rickettsia 23S rRNA nucleic acid” “methods of amplifying a Rickettsia 23S rRNA nucleic acid sequence,” “methods of amplifying a region of Rickettsia 23S rRNA nucleic acid sequence” “amplification methods,” and by other related expressions. The method embodiments can include a step of contacting a sample, which may contain Rickettsia 23S rRNA nucleic acid sequences, with one or more primers described in this document. After the contacting step, a nucleic acid amplification reaction, for example, PCR amplification (such as rRT-PCR, discussed in more detail elsewhere in this document or a different type of PCR), is performed under suitable conditions and using suitable reagents, and the amplification products can be detected by various detection procedures. The amplification methods can be used to determine if a nucleic acid sequence corresponding to Rickettsia 23S rRNA nucleic acid sequence is present in the sample, based on the detection of one or more products of the amplification.

Some other method embodiments are methods of amplifying a region of Orientia 23S rRNA nucleic acid sequence by a PCR using one or more of the primers described in this document. Such methods can be referred to as “methods of amplifying Orientia 23S rRNA nucleic acid” “methods of amplifying an Orientia 23S rRNA nucleic acid sequence,” “methods of amplifying a region of Orientia 23S rRNA nucleic acid sequence” “amplification methods,” and by other related expressions. The method embodiments can include a step of contacting a sample, which may contain Orientia 23S rRNA nucleic acid sequences, with one or more primers described in this document. After the contacting step, a nucleic acid amplification reaction, for example, PCR amplification (such as rRT-PCR, discussed in more detail elsewhere in this document or a different type of PCR), is performed under suitable conditions and using suitable reagents, and the amplification products can be detected by various detection procedures. The amplification methods can be used to determine if a nucleic acid sequence corresponding to Orientia 23 S rRNA nucleic acid sequence is present in the sample, based on the detection of one or more products of the amplification.

Some of the method embodiments rely on detection of a target region of Rickettsia and/or Orientia (which can be collectively referred to as Rickettsiaceae) 23S rRNA nucleic acid sequence using the probes according to the embodiment of the present invention in a rRT-PCR assay. One example of a method embodiment, which can be referred to as “detection method” or “method of detecting” is a method of detecting a presence or absence of Rickettsiaceae in a sample. Rickettsiaceae detection method embodiments can include a step of contacting a sample with at least one (meaning one or more) Orientia probe described in this document, a step of contacting a sample with at least one (meaning one or more) Rickettsia probe described in this document, or a step of contacting a sample with at least one Rickettsia probe and at least one Orientia probe described in this document. A method embodiment can also include a step of contacting a sample and forward and reverse primers specific for at least one nucleic acid sequence of the Orientia 23S rRNA nucleic acid sequence for which an Orientia probe according to the embodiments of the present invention is specific, and/or a step of contacting a sample and forward and reverse primers specific for at least one nucleic acid sequence of the Rickettsia 23S rRNA nucleic acid sequence for which an Orientia probe according to the embodiments of the present invention is specific. The above steps can also be combined in a single step or in multiple steps. Some of the possible combinations of primers and/or probes that can be used in the detection methods are discussed elsewhere in this document.

Some of the method embodiments rely on detection of a target region of Rickettsia 23S rRNA nucleic acid sequence using the probes according to the embodiment of the present invention in a rRT-PCR assay. One example of a method embodiment, which can be referred to as “detection method” or “method of detecting” is a method of detecting a presence or absence of Rickettsia in a sample. Rickettsia detection method embodiments can include a step of contacting a sample with a Rickettsia probe described in this document. For example, one detection method embodiment includes a step of contacting a sample with a RCKr probe described in this document. The method embodiment can also include a step of contacting a sample and forward and reverse primers specific for at least one nucleic acid sequence of the Rickettsia 23S rRNA nucleic acid sequence for which a probe according to the embodiments of the present invention is specific. A forward primer may be one of the forward primers described in this document. A reverse primer may be one of the reverse primers described in this document. Some of the possible combinations of primers and/or probes that can be used in Rickettsia detection methods are discussed elsewhere in this document.

Some of the method embodiments rely on detection of a target region of Orientia 23S rRNA nucleic acid sequence using the probes according to the embodiment of the present invention in a rRT-PCR assay. One example of a method embodiment, which can be referred to as “detection method” or “method of detecting” is a method of detecting a presence or absence of Orientia in a sample. Orientia detection method embodiments can include a step of contacting a sample with an Orientia probe described in this document. One detection method embodiment includes a step of contacting a sample with a OTSr probe described in this document. The method embodiment can also include a step of contacting a sample and forward and reverse primers specific for at least one nucleic acid sequence of the Orientia 23S rRNA nucleic acid sequence for which a probe according to the embodiments of the present invention is specific. A forward primer may be one of the forward primers described in this document. A reverse primer may be one of the reverse primers described in this document. Some of the possible combinations of primers and/or probes that can be used in Orientia detection methods are discussed elsewhere in this document.

In the detection methods that employ rRT-PCR (rRT-PCR methods or assays), rRT-PCR is performed under suitable conditions and using suitable reagents following the contacting step in order to generate a PCR cycle threshold, and this cycle threshold is compared to a threshold control cutoff value. In a semi-quantitative variation of rRT-PCR methods, if the cycle threshold is below the control cutoff value, a Rickettsiaceae (Rickettsia and/or Orientia) 23S rRNA nucleic acid sequence is present in the sample, and if the cycle threshold is above the control cutoff value, a Rickettsiaceae (Rickettsia and/or Orientia) 23S rRNA nucleic acid sequences are not detectable in the sample. An example of a cycle threshold control cutoff value is Cₜ value 40. In a quantitative variation of rRT-PCR methods, the method can include a step of determining a quantity of Rickettsiaceae (Rickettsia and/or Orientia) 23S rRNA nucleic acid sequence, when it is present in the sample. Standard curves may be generated for this purpose using known amounts of 23S rRNA nucleic acid sequence being detected.

Some of the methods according to the embodiments of the present invention include a step or steps for generating a report on the presence or absence of a Rickettsiaceae (Rickettsia and/or Orientia) in a sample. Such a report can be used for confirmation of a disease or condition with a Rickettsiaceae (Rickettsia and/or Orientia) infection, as well as selecting and/or administering a treatment, such as antibiotic. One example of such a disease or condition is RMSF. Another example of such a disease or condition is epidemic typhus. One more example of such a disease or condition is scrub typhus. The methods may comprise a step generating a report that recites a presence, absence and/or the amount Rickettsiaceae (Rickettsia and/or Orientia) detected in the sample. Based on such information, one may assess whether a subject is or has been infected in a Rickettsiaceae (Rickettsia and/or Orientia) and confirm or adjust treatment decisions, such as administration of a therapeutic agent, for example, administration of an antibiotic.

The amplification and the detection methods according to the embodiments of the present invention can have various applications. For example, they can be used in a method of determining if a human or an animal subject is or was infected with a Rickettsiaceae (Rickettsia and/or Orientia). Such a method can be used to confirm a treatment decision, for example, to determine if an appropriate treatment was administered to a patient suspected of being infected with a Rickettsiaceae bacterium (Rickettsia and/or Orientia) and/or if a treatment needs to be continued, changed or a different treatment needs to be administered (possibly in addition to an ongoing treatment). A method can also be used to conduct a Rickettsiaceae (Rickettsia and/or Orientia) surveillance, for example, to determine the amount of Rickettsiaceae (Rickettsia and/or Orientia) infections circulating in a population. In another example, testing of a collection of the samples obtained from a population using the methods of the present invention can generate more accurate epidemiological data on circulation of Rickettsiaceae (Rickettsia and/or Orientia) in a population. Embodiments of the present invention thus can provide an important contribution to public health surveillance, clinical diagnosis and scientific investigations regarding Rickettsiaceae (Rickettsia and/or Orientia) infections. The amplification and the detection methods according to the embodiments of the present invention can also be used for quality control of cell and tissue samples. For example, blood samples, particularly, but not limited, those originating from suspect sources can be tested to verify the presence and the amounts of Rickettsiaceae (Rickettsia and/or Orientia).

Systems

Systems for determining the presence, absence and/or amount of Rickettsiaceae bacteria (Rickettsia and/or Orientia) in a sample are included among the embodiments of the present invention. These systems include various stations and/or components. As used herein, the term “station” is broadly defined and includes any suitable apparatus or assemblies, conglomerations or collections of apparatuses or components suitable for carrying out the a method according to the embodiments of the present invention. The stations need not be integrally connected or situated with respect to each other in any particular way. The invention includes any suitable arrangements of the stations with respect to each other. For example, the stations need not even be in the same room. But in some embodiments, the stations are connected to each other in an integral unit.

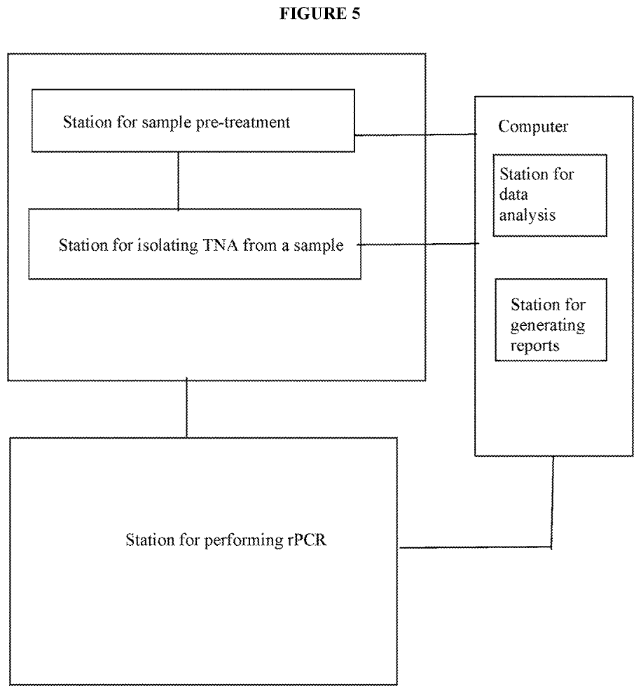

For example, and referring now to FIG. 5, a system may comprises a station for isolating TNA from a sample. A system may comprise a station for sample pre-treatment, such as centrifuging a blood sample to remove blood cells and/or adding RNAse inhibitors. The above stations may be unified as the same station or be separate stations. A system may comprise a station for performing rPCR, such as rRT-PCR. A system may comprise a station for generating reports. A system may further comprise a station or component for data analysis. The system, or parts of the system may be controlled by a computer comprising a suitably configured processor or processors and computer memory. Some parts of the system may be a computer comprising a suitably configured processor or processors and computer memory. For example, a station for data analysis and/or a station for generating reports may be a computer or controlled by a computer, as illustrated in FIG. 5.

Computers

The systems and methods according to the embodiments of the present invention can involve or use computers, computer components and/or computer-based calculations and tools. For example, the calculations and comparisons (for example, of a sample signal to a control value or range) for the methods described in this document can involve computer-based calculations and tools. In another example, the systems according to the embodiments of the present invention can be controlled by a computer- and/or include computer-based stations. In one more example, the methods according to the embodiments of the present invention can be performed using computer-based calculations and tools. In one example, a threshold value (Cₜ) employed in some of the PCR-based detection methods according to the embodiments of the present invention may be a predetermined value stored in a computer memory, and a computer can perform a comparison of a cycle threshold determined by performing rPCR according to the methods described in this document to the stored threshold value. In one more example, a computer can create or store standard curves or values for performing quantitative rPCR according to the methods described in this document and determine an amount of target nucleic acid based on the determined rPCR cycle threshold and the standard curves. In yet one more example, a computer can output the result of the determination of the presence, absence or amount of a target nucleic acid in a sample in a form of a report displayed on a computer screen or printed by a printer controlled by the computer. The above examples, are, of course, non-limiting, and a computer can perform various other functions in the embodiments of the present invention.

Computer-based tools can be advantageously provided in the form of computer programs that are executable by a general purpose computer system (which can be called “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (for example, using PERL), execution of which can be initiated by a user from a standard command line interface of an operating system such as Linux or UNIX. Commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### EXAMPLES

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### Example 1

### Total Nucleic Acid Rickettsia rRT-PCR Assay

Total nucleic acid was extracted from the banked tissue samples (“banked samples”) obtained from sixteen patients suspected of having had rickettsioses (see Table 7), thus generating total nucleic acid (TNA) (also referred to as “TNA sample”) for each of the banked samples. The banked samples from which TNA were extracted included the following samples: biopsy tissue samples listed in Table 7 (samples 18-23), which were obtained from two patients with fatal disease outcomes; and 200 μL samples of blood or serum stored at −80° C., which were obtained from the patients with both fatal and nonfatal disease outcomes. The extractions of TNA from banked tissue samples were performed using MagNA Pure® Compact instrument (Roche Diagnostics, Mannheim, Germany), following the manufacturer's protocol and using the compatible reagent kits by the same manufacturer: MagNA Pure® Compact Nucleic Acid Isolation Kit I with proteinase K and MagNA Pure® LC Total Nucleic Acid Isolation Kit Lysis/Binding Buffer for blood and serum samples and MagNA Pure LC RNA Isolation Tissue Lysis Buffer for biopsied tissue samples. TNA extracted from each of the blood and tissue samples was eluted in 200 μL of the corresponding elution buffer. TNA from each of serum samples was eluted in 50 μL of the corresponding elution buffer.

TABLE 6
Primers and probes used in RCKr assay

| Name | Sequence (5′ > 3′) | Label |
|---|---|---|
| Forward | GGT CCC ACA GAC TTA CCA AAC | None |
| Primer | TCA SEQ ID NO: 1 | |
| Reverse | TCG ACT ATG GAC CTT AGC ACC | None |
| Primer | CAT SEQ ID NO: 2 | |
| Probe | CCG AAT GTC GAT GAG TAC AGC | Fluorescein |
| | ATA GCA GAC SEQ ID NO: 3 | at 5′ end; |
| | | BHQ1 at |
| | | 3′ end |

The rRT-PCR assay was performed on each TNA sample using the probes and the primers shown in Table 6 (“RCKr assay”). The probes were labeled at the 5′-end with the reporter molecule fluorescein (FAM) and incorporated BHQ1® quencher supplied by Biosearch Technologies, Inc. (Novato, Calif.). The rRT-PCR was performed on TNA samples using 5 μL TNA and 2X PerfeCTa® Multiplex qPCR SuperMix, Low ROX or qScript XLT One-Step RT-qPCR ToughMix, Low-ROX (Quantabio, Beverly, Mass.). For comparison, rPCR assay was also performed on TNA extracted from the same samples using the primers and probes previously described in Kato et al. “Assessment of real-time PCR for detection of Rickettsia spp. and Rickettsia rickettsii in banked clinical samples.” J. Clin. Micro. 2013; 51:314-317 (“PanR8 assay”). PanR8 assay described in Kato et al. 2013 detected a target sequence in Rickettsia 505 ribosomal protein L16 (erroneously misstated in Kato et al. 2013 as 23S rRNA).

Reaction efficiency of RCKr assay was calculated to be 101.0% using samples giving five or more positive results from the 10-fold serial dilutions of TNA. PanR8 assay reaction efficiency was determined to be 95.9% using 10-fold serial dilutions of quantified stock DNA. Reaction efficiency measures the performance of a primers/probe set. Higher values (preferably closer to 100%) reflect improved performance of a primer/probe set. The comparison of the reaction efficiency values for PanR8 assay and RCKr assay showed that RCKr assay's primer and probe set had improved reaction efficiency in comparison to PanR8 assay's primer and probe set.

### Example 2

### Sensitivity and Specificity of Total Nucleic Acid Rickettsia rRT-PCR Assay

Diagnostic sensitivity and specificity of RCKr assay was determined. The results are summarized in Table 7. Both PanR8 assay and RCKr assay detected the presence of Rickettsia in all nine banked samples obtained from the patient with fatal cases of RMSF (samples 15-23), thus demonstrating 100% diagnostic sensitivity. Out of the fourteen banked blood samples obtained from the patients with nonfatal outcomes (patients 1-13, samples 1-14), the presence of Rickettsia was previously detected by PanR8 assay and recorded in six blood samples (samples 2-7), as shown in “Original Test Result” columns. Rickettsia was not previously detected in blood sample 1, which was obtained from a patient diagnosed with RMSF. In four blood samples (samples 1 and 4-6), Rickettsia was detected by both PanR8 and RCKr assays. However, RCKr assay also detected Rickettsia in three blood samples (samples 2, 3 and 7), in which PanR8 assay failed to detect Rickettsia. Both PanR8 and RCKr assays detected the absence of Rickettsia in blood samples obtained from the patients not diagnosed with a rickettsiosis (samples 8-14), which included one sample that was known to be positive for Ehrlichia chaffeensis and one sample that was known to be positive for Anaplasma phagocytophilum (samples 13 and 14). Thus, for banked blood samples, RCKr assay exhibited 100% diagnostic specificity. Table 8 shows the comparison of diagnostic sensitivity and specificity, expressed as predictive values, of RCKr and PanR8 assays conducted on blood samples. The negative predictive value for detection was increased from 70% for PanR8 assay to 100% with RCKr TNA assay, while the positive predictive values for both of the DNA and RNA assays were comparable at 100%. RCKr assay specifically detected 16 Rickettsia species, including 31 R. rickettsii, 34 R. prowazekii, and 20 R. typhi isolates (obtained from the Rickettsial Zoonoses Branch) demonstrating 100% analytical specificity when tested on isolate samples. The results of the above analytical specificity determination are illustrated in Table 9.

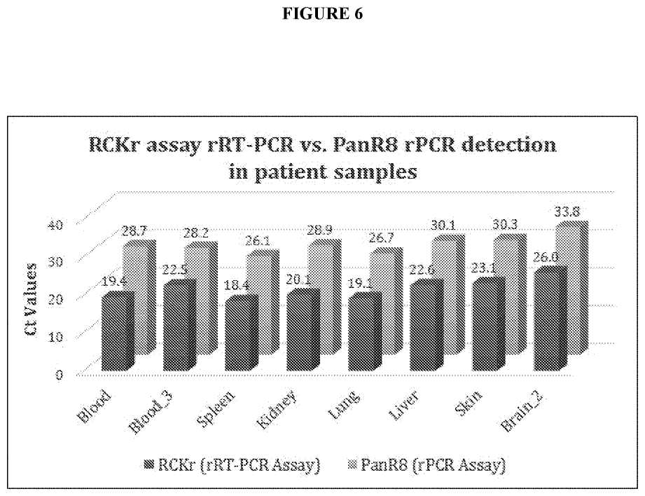

In the TNA extracted from samples obtained from the patients with fatal rickettsioses, which included blood, serum, brain, spleen, kidney, lung, liver and skin samples (samples 15-23 in Table 7), RCKr assay detected its target 23S rRNA nucleic acid sequence at an average of 7.6 (5.72-9.33) Cₜ values lower than the Cₜ values at which PanR8 assay detected its target 505 ribosomal protein L16 DNA sequence. RCKr assay detected its target nucleic acid sequence in the TNA obtained from the blood samples of the patients with non-fatal rickettsioses at an average of 5.1 (3.68-6.33) Cₜ values lower than the Cₜ values at which PanR8 assay detected its respective target sequence. FIG. 6 is a bar chart illustrating analytical sensitivity of RCKr assay using the data for samples 15-23 of Table 7.

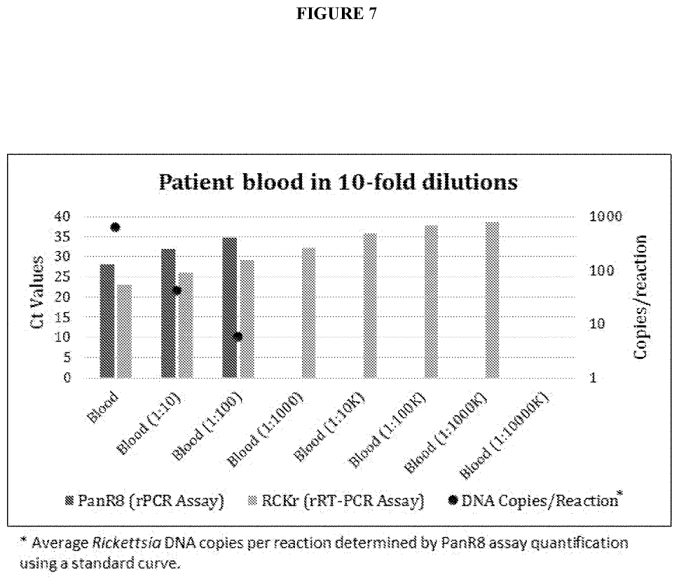

The analytical sensitivity determination for the PanR8 rPCR assay was performed using serial dilutions (10 to 10⁴ fg) of genomic R. rickettsii and R. prowazekii DNA (one isolate each). With this method, the analytical sensitivity of the PanR8 assay was determined to have DNA LOD of 8-9 genome copies (R. prowazekii and R. rickettsii, respectively) with 95% reproducibility. The analytical sensitivity of the RCKr rRT-PCR assay was performed using 10-fold serial dilutions of the TNA extracted from blood samples obtained from fatal and non-fatal patients. The results of the are summarized in Table 10. The analytical sensitivity of RCKr assay, expressed in LOD, was 100 to 100,000 times higher than analytical sensitivity of PanR8 assay. Analytical sensitivity of RCKr assay was also determined using a patient's blood sample. FIG. 7 is a bar chart illustrating analytical sensitivity of RCKr assay. A patient's blood sample was tested with the rRT-PCR assay, RCKr and the rPCR assay, PanR8 in 10-fold dilutions. The figure illustrates that RCKr assay can detect the target in this blood specimen with 10,000 times higher sensitivity than the PanR8 assay.

RCKr assay exhibited considerable and unexpectedly increased analytical sensitivity. The calculated LOD based on the analytical sensitivity testing data was that of a single Rickettsia organism per 200 μL liquid sample volume, or 5 Rickettsia per mL. Even in view of the fact that more 23 S rRNA copies than corresponding genomic DNA copies would be present in a sample, the considerable increase in analytical sensitivity is unexpected and may be due to the stability of Rickettsia rRNA.

TABLE 7
Rickettsia assay diagnostic sensitivity and specificity determination -banked samples

| | | | | | | Original test | | | | |
|---|---|---|---|---|---|---|---|---|---|---|
| | | | | | | results^{c} | | | | |
| | | | | | | | Species- | | | |
| Sample | Patient | Sample | Patient | Sample age | Patient | Rickettsia | specific | PanR8 Ct | PanR8 Copies/ | RCKr Ct |
| No. | No. | Type | outcome^{a} | (days) | diagnosis | spp. test | test | Average | reaction Average | Average |
| 1 | 1 | Blood | NF | 13 | RMSF | − | Rri− | 37.39 | 1.51 | 31.66 |
| 2 | 2 | Blood | NF | 9 | RMSF | + | Rri+ | — | — | 34.10 |
| 3 | 3 | Blood | NF | 12 | RMSF | + | Rri+ | — | — | 32.40 |
| 4 | 3 | Blood | NF | 25 | RMSF | + | Rri+ | 32.56 | 35.52 | 26.23 |
| 5 | 4 | Blood | NF | na^{b} | Epidemic typhus | + | Rpr+ | 31.05 | 92.33 | 26.30 |
| 6 | 5 | Blood | NF | 11 | Epidemic typhus | + | Rpr+ | 34.75 | 8.38 | 31.07 |
| 7 | 6 | Blood | NF | 11 | Rickettsiosis | + | Rpr− | — | — | 32.78 |
| 8 | 7 | Blood | NF | 11 | Not Rickettsiosis | − | Rpr− | — | — | — |
| 9 | 8 | Blood | NF | 8 | Not Rickettsiosis | − | Rri− | — | — | — |
| 10 | 9 | Blood | NF | 8 | Not Rickettsiosis | − | Rri− | — | — | — |
| 11 | 10 | Blood | NF | 9 | Not Rickettsiosis | − | Rri− | — | — | — |
| 12 | 11 | Blood | NF | 2 | Not Rickettsiosis | − | Rri− | — | — | — |
| 13 | 12 | Blood | NF | 4 | Ehrlichiosis | − | Ech+ | — | — | — |
| 14 | 13 | Blood | NF | na^{b} | Anaplasmosis | − | Aph+ | — | — | — |
| 15 | 14 | Serum | F | 70 | RMSF | + | Rri+ | 26.03 | 2964.11 | 18.75 |
| 16 | 14 | Blood | F | 70 | RMSF | + | Rri+ | 28.70 | 447.23 | 19.37 |
| 17 | 15 | Blood | F | 7 | RMSF | + | Rri+ | 28.23 | 619.76 | 22.52 |
| 18 | 15 | Spleen | F | 6 | RMSF | + | Rri+ | 26.07 | 2889.92 | 18.37 |
| 19 | 15 | Kidney | F | 6 | RMSF | + | Rri+ | 28.88 | 392.56 | 20.11 |
| 20 | 15 | Lung | F | 6 | RMSF | + | Rri+ | 26.71 | 1834.57 | 19.13 |
| 21 | 15 | Liver | F | 6 | RMSF | + | Rri+ | 30.08 | 166.37 | 22.58 |
| 22 | 15 | Skin | F | 6 | RMSF | + | Rri+ | 30.34 | 138.85 | 23.14 |
| 23 | 16 | Brain | F | 36 | RMSF | + | Rri+ | 33.77 | 24.18 | 26.04 |

^{a}NF = non-fatal; F = fatal
^{b}NA = data not available.
^{c}The original test results for Rickettsia spp. test were data obtain from the testing of the original extract of the specimen using PanR8 assay. The species-specific results were obtained from testing the specimen with other rPCR assays targeting agents such as R. rickettsii (Rri), R. prowazekii (Rpr), Ehrlichia chaffeensis (Ech), and Anaplasma phagocytophilum (Aph).

TABLE 8
Rickettsia assay diagnostic specificity and sensitivity

| | | |
|---|---|---|
| From Patients with | From Patients without | |
| Rickettsial Disease | Rickettsial Disease | Total |

A. PanR8 assay performed on blood samples

| | | | |
|---|---|---|---|
| Test positive | 6 | 0 | 6 |
| Test negative | 3 | 7 | 10 |
| Total | 9 | 7 | 16 |

| | |
|---|---|
| Positive predictive value (PPV)* | 100% |
| Negative predictive value (NPV)** | 70.0% |

B. RCKr assay performed on blood samples

| | | | |
|---|---|---|---|
| Test positive | 9 | 0 | 9 |
| Test negative | 0 | 7 | 7 |
| Total | 9 | 7 | 16 |

| | |
|---|---|
| Positive predictive value (PPV)* | 100% |
| Negative predictive value (NPV)** | 100% |

*PPV = number of samples from patients with disease tested positive/(number of samples from patients with disease tested positive + number of samples from patients without disease tested positive)
**NPV = number of samples from patients without disease tested negative/(number of samples from patients with disease tested negative + number of samples from patients without disease tested negative)

TABLE 9
Rickettsia assay analytical specificity

| | Number of | | |
|---|---|---|---|
| | Samples | Results | |
| Isolates | Tested | PanR8 | RCKr |
| Rickettsia species panel | 13 | Positive | Positive |
| (excluding R. rickettsii, | | | |
| R. prowazekii and R. typhi) | | | |
| Rickettsia rickettsii Isolates | 31 | Positive | Positive |
| Rickettsia prowazekii Isolates | 34 | Positive | Positive |
| Rickettsia typhi Isolates | 20 | Positive | Positive |
| Rickettsia Near Neighbors | 15 | Negative | Negative |
| Bacteria with Similar | 11 | Negative | Negative |
| Clinical Symptoms | | | |
| Human DNA - Tissues | 8 | Negative | Negative |
| Protozoan Species | 4 | Negative | Negative |
| Fungal Species | 5 | Negative | Negative |
| Bacterial Species | 27 | Negative | Negative |

TABLE 10
Rickettsia assay analytical sensitivity

| | | | RCKr | |
|---|---|---|---|---|
| | | PanR8 | | Detectable dilution |
| Sample | Patient | Lowest Predicted | Lowest Detectable | beyond expected |
| No. | No. | Detectable Dilution^{a} | Dilution (Cₜ value) | from PanR8 LOD |
| 1 | 1 | Undiluted | 1:100 (38.1) | 100 |
| 2 | 2 | Undetermined^{b} | Undiluted (34.1) | Unknown |
| 3 | 3 | Undetermined^{b} | 1:10 (36.8) | Unknown |
| 4 | 3 | 1:10 | 1:10,000 (37.3) | 1,000 |
| 5 | 4 | Undiluted | 1:10,000 (37.4) | 10,000 |
| 6 | 5 | Undiluted | 1:1,000 (38.3) | 1,000 |
| 7 | 6 | Undetermined^{b} | 1:10 (37.3) | Unknown |
| 16 | 14 | 1:10 | 1:1,000,000 (37.6) | 100,000 |
| 17 | 15 | 1:10 | 1:1,000,000 (38.5) | 100,000 |

^{a}Lowest detectable dilution was estimated based on quantification values and the dilution meeting parameters of the established PanR8 assay limit of detection (LOD) for R. prowazekii and R. rickettsii at 8 and 9 copies/reaction, respectively.
^{b}Dilution is undetermined because the recently extracted sample was negative by the PanR8 assay.

### Example 3

### Diagnostic Accuracy and Reproducibility of Rickettsia rRT-PCR Assay

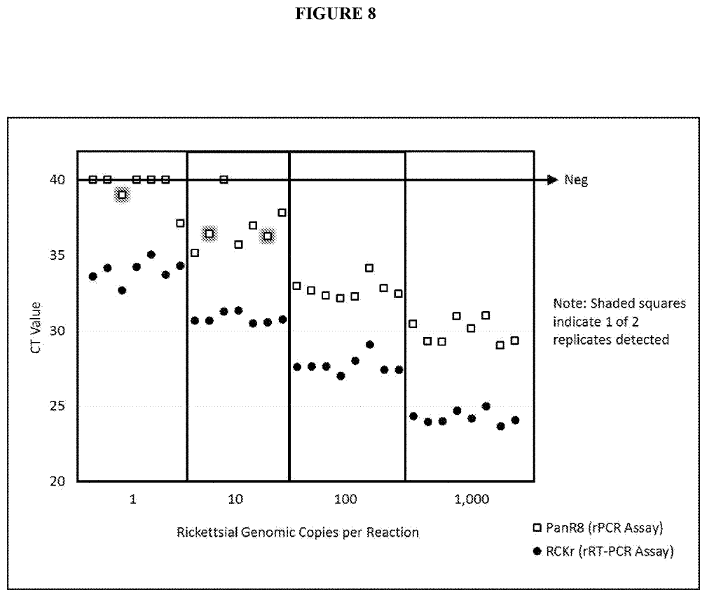

Diagnostic accuracy and reproducibility of RCKr assay were assessed. Diagnostic accuracy was determined by performing RCKr assay and PanR8 assay on TNA extracted from a contrived and blinded panel of samples in EDTA blood with high (1,000 copies per 5 μl reaction), medium (100 copies per 5 μl reaction), low (10 copies per 5 μl reaction) and equivocal (1 copy per 5 μl reaction) concentrations of Rickettsia rickettsii. The concentration of R. rickettsii in the samples was determined by quantitative PanR8 assay. The limit of detection for PanR8 assay is 8-9 copies per 5 μl reaction, thus 1 copy per 5 μl reaction was considered to be an equivocal concentration. Ten blood samples known to contain no Rickettsia organisms served as negative controls. The results of the diagnostic accuracy determination are illustrated in FIG. 8. RCKr assay produced the results consistent with the results of PanR8 assay, while at the same time demonstrating superior analytical sensitivity. RCKr assay detected R. rickettsia at 3-7 Cₜ values lower than PanR8 assay.

Intra-assay reproducibility evaluation was conducted by performing RCKr assay on TNA samples extracted from the contrived blood samples (the same panel of samples as used in the diagnostic accuracy determination). The results of intra-assay reproducibility evaluation are shown in Table 11. Inter-assay reproducibility of RCKr assay was also assessed over the course of ten assay runs using one contrived specimen with equivocal concentration, which resulted in an average Cₜ value of 33.31 and coefficient of variation of 1.97%. The low coefficient of variation demonstrated that RCKr assay was highly reproducible.

TABLE 11
Intra-assay reproducibility evaluation of RCKr assay

| | Average Cₜ | Coefficient of |
|---|---|---|
| Sample - Concentration | Value | Variation |
| 1 - Equivocal (1 copy) | 33.10 | 0.40% |
| 2 - Equivocal (1 copy) | 33.20 | 0.83% |
| 3 - High (1,000 copies) | 22.92 | 1.22% |
| 4 - High (1,000 copies) | 23.12 | 0.50% |
| 5 - Low (10 copies) | 30.12 | 0.57% |
| 6 - Low (10 copies) | 30.26 | 0.68% |
| 7 - Low (10 copies) | 30.48 | 0.90% |
| 8 - Medium (100 copies) | 26.86 | 1.49% |
| 9 - Medium (100 copies) | 26.34 | 0.61% |
| 10 - Medium (100 copies) | 26.75 | 1.60% |

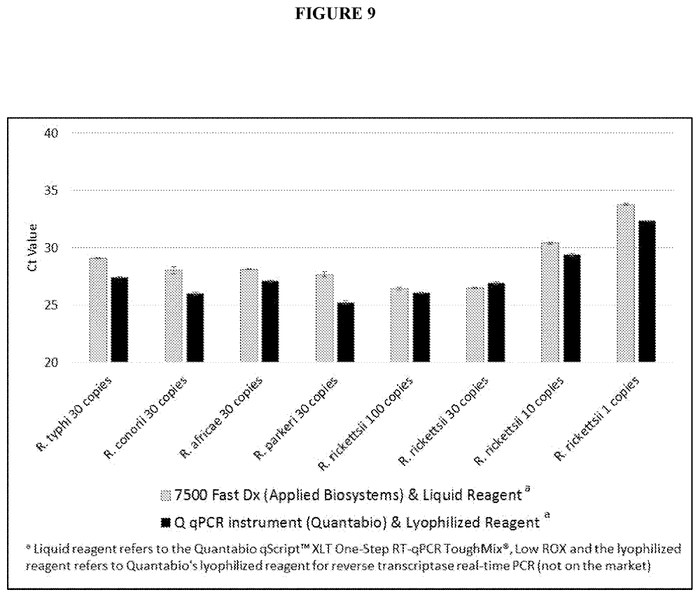

An instrument comparison of RCKr assay was performed. The results are illustrated in FIG. 9. Total nucleic acid (TNA) was extracted and quantified from antigen of a subset of Rickettsia species (R. typhi, R. conorii, R. africae, R. parkeri and R. rickettsii). RCKr assay was performed on the TNA samples using an Applied Biosystems 7500 Fast Dx instrument and a liquid reagent obtained from Quantabio (Beverly, Massachussets)—qScript XLT One-Step RT-qPCR ToughMix®, Low ROX. The TNA samples were also tested using RCKr assay on a portable Quantabio Q qPCR instrument and a proprietary lyophilized reagent mix obtained from Quantabio. The instrument comparison showed that the portable qPCR instrument used in conjunction with Quantabio proprietary lyophilized reagent generated the results comparable to those generated with the Applied Biosystems instrument and Quantabio liquid reagent, even at the lower Rickettsia levels (˜1 Rickettsia per reaction).

### Example 4

### Total Nucleic Acid Orientia tsutsugamushi rRT-PCR Assay

Total nucleic acid (TNA) was extracted from the banked tissue samples (“banked samples”) obtained from thirteen patients suspected of having had scrub typhus (see Table 13), thus generating TNA samples corresponding to each of the banked samples. The banked samples are identified in the column “Sample Type” of Table 13. The extractions of TNA by co-purification of RNA and DNA from banked eschar swab and biopsied tissue samples were performed using the QIAamp DNA Mini Kit (Qiagen USA, Germantown, Md., USA), following the manufacturer's protocol for tissue samples and a modified procedure for swab samples. The modification of the extraction method was used for the tissue and swab samples because only a single aliquot was received for DNA testing. The extracts from the QIAamp DNA Mini Kit were expected to contain both RNA and DNA, because no RNase was used in the extraction procedure. The extractions of TNA from banked blood and serum samples were performed using the MagNA Pure® Compact instrument (Roche Diagnostics, Mannheim, Germany), following the manufacturer's protocol and using the compatible reagent kits by the same manufacturer, MagNA Pure® Compact Nucleic Acid Isolation Kit I with proteinase K and MagNA Pure® LC Total Nucleic Acid Isolation Kit Lysis/Binding Buffer.

TABLE 12
Primers and probes used in OTSr assay

| Name | Sequence (5′ > 3′) | Label |
|---|---|---|
| Forward | CAA ACT CCG AAT GTC AAT AAA GT | None |
| Primer | SEQ ID NO: 8 | |
| Reverse | ATG ACG ATC TGG GCT GTT TC | None |
| Primer | SEQ ID NO: 9 | |
| Probe | TGT GGG CGC TAA GGT TCA TA | Fluorescein |
| | SEQ ID NO: 10 | at 5′ end; |
| | | BHQ1 at 3′ |
| | | end |

TABLE 13
Orientia tsutsugamushi sensitivity and specificity determination - banked samples

| Sample | Patient | Sample | Patient | Sample | Original Test | OTSr |
|---|---|---|---|---|---|---|
| No. | No. | Type | Outcome^{a} | Age (days) | Results^{b} | Average Cₜ |
| 1 | 1 | Blood | NA | 26 | — | — |
| 2 | 2 | Blood | NF | 3 | — | — |
| 3 | 3 | Serum | NF | 14 | — | — |
| 4 | 4 | Blood | NF | 2 | — | — |
| 5 | 5 | Blood | NF | 8 | — | — |
| 6 | 5 | Blood | NF | 8 | — | — |
| 7 | 6 | Blood | NF | 54 | — | — |
| 8 | 7 | Blood | NA | 3 | — | — |
| 9 | 7 | Serum | NA | 3 | — | — |
| 10 | 8 | Serum | F | 10 | — | — |
| 11 | 8 | Tissue | F | 10 | — | — |
| 12 | 8 | Blood | F | 9 | — | — |
| 13 | 8 | Blood | F | 9 | — | — |
| 14 | 9 | Blood | NF | 7 | — | — |
| 15 | 9 | Blood | NF | 7 | — | — |
| 16 | 10 | Blood | NF | 10 | — | — |
| 17 | 11 | Tissue | NF | 6 | 27.46 | 25.17 |
| 18 | 12 | Serum | NF | 12 | 40.16^{c} | 35.34 |
| 19 | 13 | Eschar swab | NF | 3 | 38.31 | 34.31 |
| 20 | 13 | Serum | NF | 21 | 38.88 | 34.40 |

^{a}NF = non-fatal; F = fatal; NA = data not available
^{b}Original test results were based on the data obtained from the samples tested with an alternative rPCR assay (“Ori5” assay), with the exception of samples 1 and 2 that were tested by GroESL nested PCR assay
^{c}Patient confirmed for O. tsutsugamushi infection by serology testing

The rRT-PCR assay was performed on each TNA sample using the probes and the primers shown in Table 12 (“OTSr assay”). The probes were labeled at the 5′-end with the reporter molecule fluorescein (FAM) and incorporated BHQ1™ quencher supplied by the Division of Scientific Resources, Biotechnology Core Facility Branch, Center for Disease Control (CDC), USA. The rRT-PCR was performed on TNA samples using 5 μL TNA and 2X PerfeCTa® Multiplex qPCR SuperMix, Low ROX® or qScript XLT 1-Step RT-qPCR ToughMix Low-ROX (Quanta Biosciences). For comparison, an alternative rPCR assay (“Ori5” assay) was performed on the TNA extracted from the same samples using the forward primer in the groESL operon region previously described in Kelly et al. “Detection and characterization of Rickettsia tsutsugamushi (Rickettsiales: Rickettsiaceae) in infected Leptotrombidium (Leptotrombidium) fletcheri chiggers (Acari: Trombiculidae) with the polymerase chain reaction.” J Med Entomol. 31:691-699 (1994), with a newly designed reverse primer and probe. Samples 1 and 2 were tested by GroESL nested PCR assay using the primers described in Kelly et al. (1994) and Mettille et al. “Reducing the risk of transfusion-transmitted rickettsial disease by WBC filtration, using Orientia tsutsugamushi in a model system.” Transfusion 40(3):290-296 (2000), as indicated in footnote b to Table 13. The results summarized in Table 13 showed that OTSr assay accurately detected Orientia in diagnostic specimens with higher analytical sensitivity—at an average of 3.9 (2.28-4.82) Cₜ values lower—than Ori5 assay.

TABLE 14
Comparative analytical specificity of OTSr and Ori5 assays

| | Number of | | |
|---|---|---|---|
| | Samples | Results | |
| Isolates | Tested | OTSr | Ori5 |
| Orientia tsutsugamushi isolate panel | 17 | Positive | Positive |
| Rickettsiaceae other than Orientia (Ehrlichia chaffeensis, | 7 | Negative | Negative |
| Anaplasma phagocytophilum, Rickettsia typhi, R. rickettsii, | | | |
| R. africae, Coxiella burnetii, Bartonella Quintana') | | | |
| Background Clinical and Environmental Bacteria (Escherichia | 9 | Negative | Negative |
| coli Migula, Proionibacterium acnes Gilchrist, Enterococcus | | | |
| faecalis Portland, Staphylococcus aureus Serotype 3, | | | |
| Staphylococcus aureus Seattle 1945, Neisseria meningitidis | | | |
| FAM 18, Staphylococcus epidermidis PCI 1200, | | | |
| Staphylococcus epidermidis Evans, Streptococcus pyogenes | | | |
| Lancefield's group A) | | | |

TABLE 15
Comparative analytical sensitivity of OTSr and Ori5 assays
with respect to O. tsutsugamushi isolate panel

| | | OTSr Assay Average Ct | | Ori5 Assay Average Cₜ | |
|---|---|---|---|---|---|
| | | 10,000 | | 10,000 | |
| Orientia tsutsugamushi | | copies per | 30 copies | copies per | 30 copies per |
| Isolate | Origin | reaction | per reaction | reaction | reaction |
| AFC3 | Thailand | 15.20 | 24.27 | 24.27 | 31.26 |
| AFSC7 | Thailand | 15.27 | 24.46 | 24.17 | 32.26 |
| Boryong | S. Korea | 15.23 | 24.39 | 24.20 | 32.54 |
| Brown | Australia | 14.87 | 23.97 | 24.67 | 33.56 |
| BSE125 | Solomon | 15.25 | 24.10 | 27.45 | 35.89 |
| | Islands | | | | |
| Calcutta | India | 15.35 | 24.27 | 27.01 | 35.92 |
| Domrow | Australia | 15.97 | 24.96 | 26.87 | 35.22 |
| Farfan | Vietnam | 15.09 | 24.23 | 24.79 | 33.20 |
| H9780 | Malaysia | 14.68 | 23.88 | 23.93 | 31.21 |
| JC531 | Pakistan | 14.87 | 23.89 | 24.67 | 34.08 |
| Jpn5 | Japan | 15.28 | 24.38 | 25.47 | 34.32 |
| MAKI 19 | Taiwan | 15.63 | 24.70 | 26.51 | 35.36 |
| R25 | China | 14.38 | 23.41 | 26.66 | 34.63 |
| TH1811 | Thailand | 15.65 | 24.89 | 24.36 | 32.68 |
| Kato* | Prototype | 16.33 | 25.15 | 27.69 | 35.89 |
| | strain | | | | |
| Karp* | Prototype | 14.75 | 23.54 | 27.76 | 36.46 |
| | strain | | | | |
| Gilliam* | Prototype | 14.49 | 23.39 | 27.73 | 36.40 |
| | strain | | | | |

*Reference standard prototype strains of O. tsutsugamushi.

### Example 5

### Sensitivity, Specificity and Reproducibility of Orientia tsutsugamushi rRT-PCR Assay

Comparative analytical specificity and sensitivity of OTSr and Ori5 assays was determined. The results are summarized in Tables 14 and 15. The results of the analytical specificity determination summarized in Table 14 showed that OTSr assay was specific and accurately detected different O. tsutsugamushi isolates, while excluding various Rickettsiaceae other than Orientia, background clinical bacteria and background environmental bacteria. OTSr assay results were comparable to those generated by Ori5 assay. The results of the analytical sensitivity determination summarized in Table 15 showed that OTSr assay was able to detect seventeen different O. tsutsugamushi isolates originating from various countries at 10,000 and 30 copies per reaction. The results demonstrated superior analytical sensitivity of OTSr assay, which detected Orientia at an average of 10.3 (6.99-13.25) Cₜ values lower than the Ori5 assay.

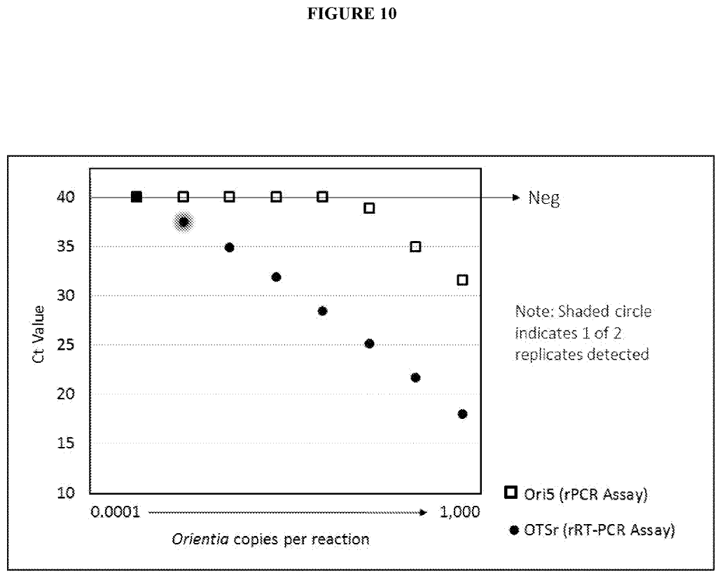

Comparative analytical sensitivity determination of OTSr and Ori5 assays was also performed using O. tsutsugamushi samples of known concentrations. The results are illustrated in FIG. 10. Quantified O. tsutsugamushi TNA was tested at a range of 10-fold dilutions—from 1,000 copies to 0.0001 copy per reaction—using Ori5 and OTSr assays. OTSr assay exhibited higher sensitivity, expressed as lower LOD, than Ori5 assay. While Ori5 assay exhibited LOD of 10 O. tsutsugamushi copies per reaction, OTSr assay detected Orientia at 0.001 copy per reaction, which is a calculated theoretical value based on 10-fold dilutions.

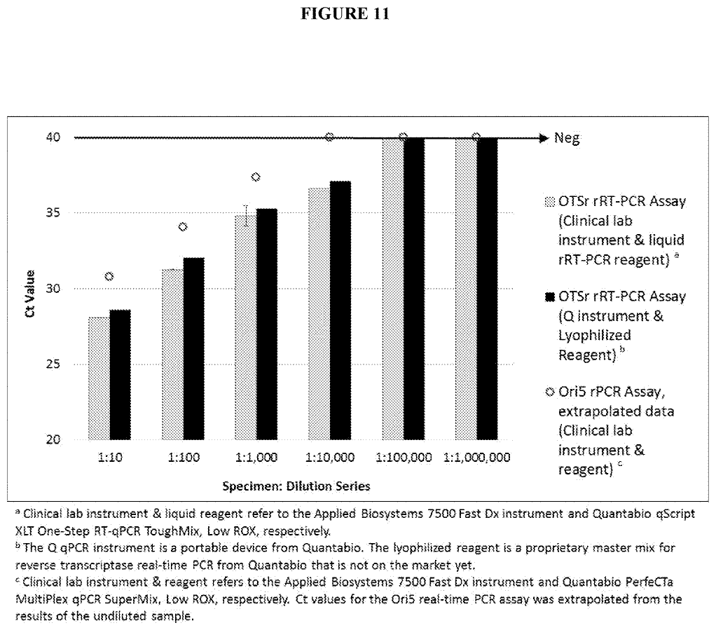

Instrument comparison of OTSr assay was performed using serial dilutions of O. tsutsugamushi-positive clinical specimen. The results are illustrated in FIG. 11. A confirmed O. tsutsugamushi-positive tissue sample was tested using OTSr assay at a range of 10-fold dilutions. Sample dilutions were tested using OTSr assay performed on Applied Biosystems 7500 Fast Dx instrument with the liquid reagent obtained from Quantabio—qScript XLT One-Step RT-qPCR ToughMix, Low ROX, as well as on a portable Quantabio Q qPCR instrument with the proprietary lyophilized reagent mix provided by Quantabio. The testing demonstrated that OTSr assay performed on portable Q qPCR instrument with lyophilized reagent produced the results comparable to those of OTSr assay formed on Applied Biosystems 7500 Fast Dx instrument with the liquid reagent. In addition, OT Sr results were compared to data points that were extrapolated or calculated (a 10-fold dilution is represented by a 3 Cₜ value change) based on the undiluted Cₜ value produced by the Ori5 assay. The comparison demonstrated superior analytical sensitivity of OTSr assay, which detected Orientia at lower Cₜ values than Ori5 assay.

All patents, patent applications, publications, and abstracts cited above are incorporated herein by reference in their entirety. Various embodiments of the invention have been described in fulfillment of the various objectives of the invention. It should be recognized that these embodiments are merely illustrative of the principles of the present invention. Numerous modifications and adaptations thereof will be readily apparent to those of skill in the art without departing from the spirit and scope of the invention as defined in the following claims.

1. A method of detecting presence or absence of a Rickettsiaceae bacterium in a sample, comprising performing on a total nucleic acid isolated from the sample a real-time reverse transcriptase polymerase chain reaction (rRT-PCR) to detect a target nucleic acid sequence characteristic of the Rickettsiaceae bacterium.

2. The method of claim 1, further comprising isolating the total nucleic acid from the sample prior to performing rRT-PCR.

3. The method of claim 1, wherein the sample is obtained from a patient suspected of being infected with Rickettsia and/or Orientia.

4. The method of claim 1, wherein the sample is obtained from a patient suspected of having a rickettsiosis or scrub typhus.

5-8. (canceled)

9. The method of claim 1, wherein the target nucleic acid sequence is conserved across Rickettsia species being detected.

10. The method of claim 1, wherein the method detects a Rickettsia species pathogenic to humans is detected, and wherein the target nucleic acid sequence is conserved across the Rickettsia species pathogenic to humans.

11. The method of claim 1, wherein the method detects Orientia species pathogenic to humans.

12. The method of claim 1, wherein the target nucleic acid sequence is a 23S rRNA nucleic acid sequence.

13. The method of claim 1, wherein the rRT-PCR is performed using a pair of primers, at least one of which comprises SEQ ID NO:1, SEQ ID NO:2, SEQ ID NO:4 or SEQ ID NO:5; and/or
the rRT-PCR is performed using a probe comprising SEQ ID NO:3, SEQ ID NO:6 or SEQ ID NO:7; and/or
the rRT-PCR is performed using a pair of primers, at least one of which comprises SEQ ID NO:8, SEQ ID NO:9, SEQ ID NO:11, SEQ ID NO:12, SEQ ID NO:14 or SEQ ID NO:15; and/or
the rRT-PCR is performed using a probe comprising SEQ ID NO:10, SEQ ID NO:13, SEQ ID NO:16, SEQ ID NO:17, SEQ ID NO:18, SEQ ID NO:19 or SEQ ID NO:20.

14-17. (canceled)

18. A method of assessing a Rickettsiaceae infection status of a patient, comprising:
performing on a total nucleic acid isolated from a sample obtained from the patient a real-time reverse transcriptase polymerase chain reaction (rRT-PCR) to detect at least one target nucleic acid sequence characteristic of Rickettsiaceae, wherein when the at least one target nucleic acid sequence is detected, the status of the patient is infected with Rickettsiaceae, and when the target nucleic acid sequence is not detected, the status of the patient is not infected with Rickettsiaceae.

19. The method of claim 18, further comprising administering a treatment to the patient based on the assessed Rickettsiaceae infection status of the patient.

20. The method of claim 18, further comprising a step of generating a report indicating the assessed Rickettsiaceae infection status of the patient.

21. The method of claim 18, wherein the Rickettsiaceae infection is a Rickettsia infection or an Orientia infection.

22. A probe comprising an oligonucleotide comprising a sequence at least 90% identical to SEQ ID NO:3, SEQ ID NO:6, SEQ ID NO:7, SEQ ID NO:10, SEQ ID NO:13, SEQ ID NO:16, SEQ ID NO:17, SEQ ID NO:18, SEQ ID NO:19 or SEQ ID NO:20 linked to at least one of a fluorophore moiety and a quencher moiety.

23-34. (canceled)

35. A kit for detecting a Rickettsiaceae nucleic acid sequence in a sample, comprising at least one probe of claim 18 and other reagents for performing an rRT-PCR assay.

36. (canceled)

37. The kit of claim 35, wherein the other reagents comprise at least one primer comprising a sequence at least 90% identical to a sequence selected from the group consisting of SEQ ID NO:1, SEQ ID NO:2, SEQ ID NO:4, and SEQ ID NO:5.

38-43. (canceled)

44. A primer comprising an oligonucleotide having a sequence at least 90% identical to a SEQ ID NO:1, SEQ ID NO:2, SEQ ID NO:4, SEQ ID NO:5, SEQ ID NO:8, SEQ ID NO:9, SEQ ID NO:11, SEQ ID NO:12, SEQ ID NO:14 or SEQ ID NO:15.

45-51. (canceled)

52. A kit for amplifying a nucleic acid sequence of a region of Rickettsiaceae 23S rRNA nucleic acid sequence, comprising at least one primer of claim 44 and one or more other ingredients for performing a PCR.

53-54. (canceled)

55. A method of amplifying a nucleic acid sequence, comprising:
contacting the sample with at least one primer of claim 44; and,
performing a PCR.

56. A method of detecting a nucleic acid comprising a region of Rickettsiaceae 23 S rRNA nucleic acid sequence in the sample, comprising:
performing the method of claim 55; and,
detecting one or more products of the amplification,
wherein the nucleic acid is present in the sample if the one or more products of the amplification are detected.

57. A system for detecting presence or absence of one or more Rickettsiaceae in a sample, comprising performing on a total nucleic acid isolated from the sample a real-time reverse transcriptase polymerase chain reaction (rRT-PCR) to detect a target nucleic acid sequence characteristic of the one or more Rickettsiaceae, the system comprising:
optionally, a station for isolating the total nucleic acid from the sample;
a station for performing rRT-PCR on the isolated total nucleic acid to detect a target nucleic acid sequence characteristic of the one or more Rickettsiaceae;
and, optionally, a station for generating a report on the presence or absence of Rickettsiaceae in the sample based on detected presence or absence of the target nucleic acid sequence.

58-59. (canceled)
